(19) United States
(12) Patent Application Publication
(10) Pub. No.: US 2023/0159484 A1
(43) Pub. Date: May 25, 2023

(54) FLUORESCENT DYES HAVING HIGH STOKES SHIFT, ON THE BASIS OF BRIDGED BENZOPYRYLIUM SALTS

(71) Applicant: DYOMICS GmbH, Jena (DE)

(72) Inventors: Bernd SCHWEDER, Jena (DE); Frank LEHMANN, Jena (DE); Matthias WENZEL, Jena (DE); Peter CZERNEY, Weimar (DE)

(21) Appl. No.: 17/995,356

(22) PCT Filed: Apr. 1, 2021
(86) PCT No.: PCT/EP2021/058647

§ 371 Date: Oct. 3, 2022

(30) Foreign Application Priority Data
Apr. 3, 2020 (DE) 10 2020 109 362.4

(51) Int. Cl. C07D 311/86; C09B 57/02; C09B 57/00; C07D 311/88; C07C 43/00; C07D 311/82; C07D 405/12; C07D 491/052; C07D 405/14; C07D 405/06; C07D 213/46; C07D 405/04; C07D 493/04

(52) U.S. Cl. CPC C07D 311/86; C09B 57/02; C09B 57/00; C07D 311/88; C07C 43/00; C07D 311/82; C07D 405/12; C07D 491/052; C07D 405/14; C07D 405/06; C07D 213/46; C07D 405/04; C07D 493/04

(57) ABSTRACT

The invention relates to novel, water-soluble fluorescent dyes with high fluorescence quantum yield based on oxygen-containing heterocycles, their reactive derivatives and dye conjugates, and their use for labelling samples and detecting analytes. The compounds of the new dye class are compatible with commercial excitation light sources and are characterised by Stokes shifts of more than 50 nm.

### STATE OF THE ART IN SCIENCE AND RESEARCH

Fluorescence-based markers have been used for decades in biological, biotechnological and medical research as well as in medical diagnostics [Brinkley M., A Brief Survey of Methods for Preparing Protein Conjugates with Dyes, Haptens and Cross-Linking Reagents, Bioconjugate Chem, 3 (1992) 2-12; Waggoner A., Covalent Labeling of Proteins and Nucleic Acids with Fluorophores, Meth. Enzymol., 246 (1995) 362-373; Hermanson G. T., Bioconjugate Techniques, Academic Press 2013; Springer Series on Fluorescence 18, 2019 (Pedras B. Editor): Fluorescence in Industry]. Originally, chromophores known from the field of textile and sensitizer dyes were derivatised in order to optimise them for applications in the aqueous physiological milieu. Particular focal points of the developments here were water solubility as well as a high quantum yield in aqueous solution.

The striving for ever higher information gain in bioanalytical methods finds its expression in multi-colour analyses, such as those by now belonging to everyday laboratory work in flow cytometry [Lee L. G., NEAR-IR Dyes in Three Color Volumetric Capillary Cytometry: Cell Analysis With 633- and 785-nm Laser Excitation, Cytometry, 21 (1995) 120-128], DNA sequencing and various PCR methods (Roche's LightCycler).

Also in high-resolution optical microscopy, especially STED microscopy [Sednev M. V., Belov V. N., Hell S. W., Fluorescent dyes with large Stokes shifts for super-resolution optical microscopy of biological objects: a review, MethodsAppl. Fluoresc. 3 (2015) dyes with high Stokes shifts are used. Initially, dye combinations were used here that provide spectrally distinguishable signals by means of energy transfer from one donor to different acceptors when excited by one and the same (monochromatic) light source. Examples of this are the DNA sequencers from Amersham (now GE Healthcare) and ABI (now Life Technologies), which came onto the market in the late 1990s.

An alternative approach for multiplex applications is the use of dyes that allow spectral differentiation without energy transfer between donor and acceptor. Examples of this can be found in the MegaStokes dyes known since about 2002 (EP 1 318 177 B1, EP 1 535 969 B1). These are preferably tailored to an excitation wavelength between 470 nm (blue-green LED) and 500 nm (488 nm Ar-ion laser), as they thus best corresponded to the state of the art in excitation light sources at that time.

The combination of several excitation light sources with more than one chromophore per light source has now also found its way into the state of the art (Solexa, WO 2007/135 368 A2). A typical application here is Next Generation Sequencing (NGS).

With the availability of light-intensive, short-wave excitation sources such as UV-LEDs or violet laser diodes, the basis of suitable fluorophores for bioanalytical applications has also broadened. Examples include the Pacific Orange from Molecular Probes (U.S. Pat. No. 8,158,801) and a series of benzoxazole-based dyes from Dyomics (U.S. Pat. No. 9,453,010 B2, EP 2 886 542 B1), which allowed multicolour analyses in analogy to the MegaStokes™ dyes, but at an excitation around 400 nm.

The invention has the aim of making fluorescent markers based on bridged benzopyrylium compounds accessible, in which molecules K to be labelled can be bound via the linker L and the reactive group A, and the fluorescent marker has as many of the following properties as possible: large Stokes shift, high photostability and storage stability, solubility in aqueous media and high fluorescence quantum yields.

The invention describes compounds (in particular bridged benzopyrylium salts) of the general formula 1

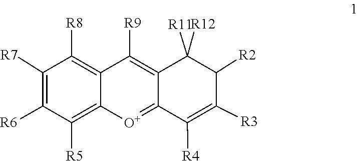

and salts as well as solvates thereof, wherein
R11 and R12 are each independently of the other hydrogen or alkyl, alkyl preferably being C₁-C₄ alkyl, particularly preferably methyl,
R2 is hydrogen, alkyl, preferably C₁-C₄-alkyl, or alkenyl, hydrogen being preferred in one embodiment,
R3 is hydrogen, alkyl, preferably C₁-C₄-alkyl, particularly preferably methyl, aryl, hydroxy or oxo, alkoxy, preferably methoxy or ethoxy, particularly preferably ethoxy, aryloxy, NR18R19, or a group Q,
wherein R18 and R19 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, more preferably ethyl, (vii) ω-sulfonic acid alkyl (—(CH₂)ₓ—SO₃⁻), wherein x is preferably 1-5, particularly preferably 3, (viii) ω-carboxylic acid alkyl (—(CH₂)_{y}—CO₂H), wherein y is preferably 1-8, particularly preferably 6, and (ix) ethyl esters of (viii),
and wherein NR18R19 is particularly preferably selected from 3-aminopropanesulfonate, N-methylaniline, 4-(methylamino)benzenesulfonate, aniline, 5-carboxypentylamine and 3-carboxypropyl(methyl)amine,
and Q is a heterocyclic structure selected from a structure of formula 2 or 3

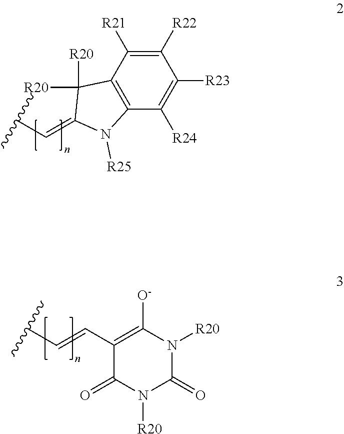

where n=1, 2 or 3; wherein each R20 is independently of the other alkyl, preferably C₁-C₈, alkyl, ω-sulfonic acid alkyl (—(CH₂)ₓ—SO₃⁻), wherein x is preferably 1-5, particularly preferably 3, or a reactive group A bound via a linker L, R21, R22, R23, R24 are each independently of the other hydrogen, a sulfonic acid or a sulfonic acid derivative, R25 is hydrogen, alkyl, preferably C₁-C₈ alkyl, ω-sulfonic acid alkyl (—(CH₂)ₓ—SO₃⁻), where x is preferably 1-5, particularly preferably 3, or a reactive group A bound via a linker L,
or R2 and R3 are bridged to form a saturated ring, partially unsaturated ring, aromatic ring or heteroaromatic ring together with the carbon atoms to which they are bound, which ring may contain further substituents,
R4 is hydrogen, bromine, chlorine, sulfonic acid or a sulfonic acid derivative, alkyl, aryl or heteroaryl, hydrogen, sulfonic acid and pyridine (4-pyridyl) being preferred,
or R3 and R4 are bridged to form a saturated ring, partially unsaturated ring, aromatic ring or heteroaromatic ring together with the carbon atoms to which they are bound, which ring may contain further substituents, in particular a sulfonic acid or sulfonic acid derivative,
R5 is hydrogen, sulfonic acid or a sulfonic acid derivative, hydrogen being preferred,
R6 is hydrogen, bromine, chlorine, hydroxy, alkoxy (for example 6-oxy-hexanoic acid), aryloxy or NR29R30, wherein R29 and R30 are each independently of the other hydrogen, alkyl, aryl or a reactive group A bound via a linker L, wherein R29 and R30 are each independently of the other preferably selected from (i) C₁-C₄ alkyl, preferably ethyl, (ii) ω-sulfonic acid alkyl (—(CH₂)ₓ—SO₃⁻), where x is preferably 1-5, particularly preferably 3, (iii) ω-carboxylic acid alkyl (—(CH₂)_{y}—CO₂H), where y is preferably 1-8, particularly preferably 6, and (iv) ethyl esters of (iii),
or R5 and R6 are bridged to form a saturated ring, partially unsaturated ring, aromatic ring or heteroaromatic ring together with the carbon atoms to which they are bound, which ring may contain further substituents, in particular a sulfonic acid or sulfonic acid derivative,
R7 is hydrogen, bromine, chlorine, hydroxy, alkoxy, aryloxy, NR31R32, sulfonic acid or a sulfonic acid derivative, hydrogen, sulfonic acid or a sulfonic acid derivative being particularly preferred, wherein R31 and R32 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, more preferably ethyl, (vii) ω-sulfonic acid alkyl (—(CH₂)ₓ—SO₃⁻), where x is preferably 1-5, particularly preferably 3, (viii) ω-carboxylic acid alkyl (—(CH₂)_{y}—CO₂H), where y is preferably 1-8, particularly preferably 6, and (ix) ethyl esters of (viii),
or R6 and R7 are bridged to form a saturated ring, partially unsaturated ring, aromatic ring or heteroaromatic ring together with the carbon atoms to which they are bound, which ring may contain further substituents, in particular a sulfonic acid or sulfonic acid derivative,
R8 is hydrogen, methyl or ethyl, hydrogen being preferred,
or R7 and R8 are bridged to form a saturated ring, partially unsaturated ring, aromatic ring or heteroaromatic ring together with the carbon atoms to which they are bound, which ring may contain further substituents, in particular a sulfonic acid or a sulfonic acid derivative,
R9 is hydrogen, alkyl, preferably C₁-C₆ alkyl, or 2-carboxyphenyl, hydrogen being preferred,
L is a linker selected from —(CH₂)ₛ— and —[(CH₂)ₘ—O]ₚ—(CH₂)ₘ—, where m is an integer from 2-5 and p and s are each independently of the other an integer from 1-10, wherein
each compound contains no linker L or a linker L having a reactive group A bound to L for covalent bonding to a molecule K to be labelled, wherein
A is an amine (—NH₂), hydroxy (—OH) or phosphoramidite (—O—P—[O—CH₂—CH₂—CN]—N[(CH(CH₃)₂]₂) function, a carboxylic acid (—COOH), an alkyl ester or active ester derived therefrom (NHS ester, sulfo-NHS ester, tetrafluoro-phenyl ester, p-sulfo-tetrafluoro-phenyl ester), a carboxylic acid hydrazide (—CONHNH₂) or a carboxylic acid amide (—CONHR28) with R28 equal to —(CH₂)ₜ—Y, where
Y is —OH, —NH₂, —NH₃⁺, maleimide (—N[CO—CH]₂), —NCS, —NCO, —NH—CO—CH₂—I, —NH—CO—CH₂—Br, -azide (—N₃), -alkyne (—CCH) or -phosphoramidite (—O—P—[O—CH₂—CH₂—CN]—N—[CH—(CH₃)₂]₂) and t is an integer from 1-10, and
K is a component selected from the group consisting of haptens (molecules which represent an incomplete antigen and exhibit the effect of an antigen only when bound to proteins or cell structures), proteins, antibodies (proteins which are formed in response to antigens), low-molecular-weight drugs (active constituents in drugs which, because of their relatively low molar mass of up to about 800 g/mol, in contrast to, for example, proteins as very large molecules, are able to penetrate cells), peptides (small or short-chain proteins up to about 100 linked amino acids), nucleotides (basic building blocks of nucleic acids such as DNA or RNA, which consist of a phosphate part, a monosaccharide part and a nucleobase part such as adenine, guanine, cytosine, thymine or uracil), nucleosides (basic building blocks of nucleic acids such as DNA or RNA, which do not have a phosphate part, but consist only of a monosaccharide part and a nucleobase part), DNA oligomers (in contrast to DNA as a macromolecule, molecules of deoxyribonucleic acid with a relatively small, not exactly defined number of nucleotides), polymers (synthetic or natural, chain-like or branched chemical compound consisting of repeating units, the monomers; polymers may also as copolymers consist of at least two different monomers in different proportions and arrangements).

Preferably, aryl substituents and/or heteroaryl substituents (as in R3, R4, R6, R7, R18, R19, R29, R30, R31, R32) contain further substituents such as sulfonic acids or sulfonic acid derivatives and/or alkoxy groups and/or substituted amino groups.

Preferably, a compound according to the invention contains at least one group selected from a sulfonic acid group, a sulfonic acid derivative, an alkoxy group and an amino group, preferably a sulfonic acid group.

The term sulfonic acid also includes the term sulfonate and the term sulfonate also includes the term sulfonic acid. The term carboxylic acid also includes the term carboxylate and the term carboxylate also includes the term carboxylic acid.

The verbs “comprise” and “contain” and their conjugations also comprise the verb “consist of” and its conjugations.

Preferred embodiments are also given in the dependent claims.

Preferably, the compound according to the invention exhibits fluorescence.

Preferably, the compound according to the invention is a fluorescent dye. In other words, preferably the compound is suitable as a fluorescent dye.

In one embodiment, R3=hydroxy.

In the case R3=hydroxy, depending on the pH, the bridged compounds are present as 3-hydroxy-xantheneium salts 1 at low pH values and as 3-oxo-2H-xanthenes 4 with neutral basic body at higher pH values. In one embodiment, R3 is preferably hydroxy, the compound being present as neutral basic body 3-oxo-2H-xanthene 4 depending on the pH-value.

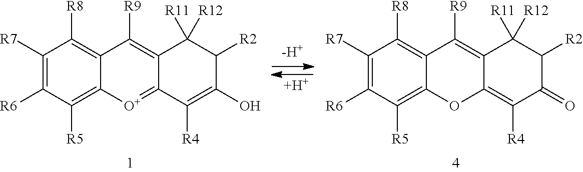

In compounds according to the invention, one or more selected from R2-R3, R3-R4, R5-R6, R6-R7 and R7-R8 may be bridged by forming saturated rings, partially unsaturated rings, aromatic rings or heteroaromatic rings which independently of the other contain further substituents, in particular sulfonic acids or sulfonic acid derivatives.

In one embodiment, at least one pair selected from R3 is bridged with R4, R5 is bridged with R6, R6 is bridged with R7 and R7 is bridged with R8 to form a saturated ring, partially unsaturated ring, aromatic ring or heteroaromatic ring together with the carbon atoms to which they are bound, wherein each ring may independently of the other contain further substituents, in particular sulfonic acids or sulfonic acid derivatives.

In one embodiment, R3 and R4 are not bridged together in such a way that they form, together with the carbon atoms to which they are bound, any ring selected from a saturated ring, partially unsaturated ring, aromatic ring or heteroaromatic ring.

In one embodiment, R3 and R4 are not bridged together in such a way that, together with the carbon atoms to which they are bound, they form an aromatic ring.

Furthermore, compounds according to the invention are indicated by formula 5 (chromeno-xantheneium compounds), to which, for example, a bridging of R2-R3 leads.

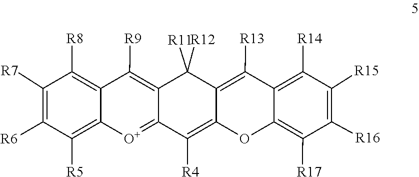

In addition to the general explanations, the following applies to the additional substituents R13-R17

R13 is hydrogen, alkyl, preferably C₁-C₄ alkyl, or 2-carboxyphenyl, hydrogen being particularly preferred,
R14 is hydrogen or alkyl, preferably C₁-C₄-alkyl, hydrogen being particularly preferred,
R15 is hydrogen, bromine, chlorine, hydroxy, alkoxy, aryloxy, NR33R34, sulfonic acid or a sulfonic acid derivative or bridged to R16, hydrogen being particularly preferred,
wherein R33 and R34 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, more preferably ethyl, (vii) ω-sulfonic acid alkyl (—(CH₂)ₓ—SO₃⁻), where x is preferably 1-5, particularly preferably 3, (viii) ω-carboxylic acid alkyl (—(CH₂)_{y}—CO₂H), where y is preferably 1-8, particularly preferably 6, and (ix) ethyl esters of (viii),
or R14 and R15 are bridged to form a saturated ring, partially unsaturated ring, aromatic ring or heteroaromatic ring together with the carbon atoms to which they are bound, which ring may contain further substituents, in particular a sulfonic acid or sulfonic acid derivative,
R16 is hydrogen, bromine, chlorine, hydroxy, alkoxy, aryloxy or NR35R36, with NR35R36 being particularly preferred,
wherein R35 and R36 are each independently selected from (i) hydrogen, (ii) benzyl, (iii) aryl, (iv) heteroaryl, (v) a reactive group A bound via a linker L, (vi) alkyl, preferably C₁-C₄ alkyl, more preferably ethyl, (vii) ω-sulfonic acid alkyl (—(CH₂)x-SO₃⁻), where x is preferably 1-5, particularly preferably 3, (viii) ω-carboxylic acid alkyl (—(CH₂)_{y}—CO₂H), where y is preferably 1-8, particularly preferably 6, and (ix) ethyl esters of (viii) are selected,
it being particularly preferred that R35 and R36 are each independently of the other preferably selected from C₁-C₄ alkyl, preferably ethyl, and ω-carboxylic acid alkyl (—(CH₂)_{y}—CO₂H), wherein y is preferably 1-8, particularly preferably 6,
or R15 and R16 are bridged to form a saturated ring, partially unsaturated ring, aromatic ring or heteroaromatic ring together with the carbon atoms to which they are bound, which ring may contain further substituents, in particular a sulfonic acid or sulfonic acid derivative,
R17 is hydrogen, sulfonic acid or a sulfonic acid derivative, hydrogen being particularly preferred,
or R16 and R17 are bridged to form a saturated ring, partially unsaturated ring, aromatic ring or heteroaromatic ring together with the carbon atoms to which they are bound, which ring may contain further substituents, in particular a sulfonic acid or sulfonic acid derivative.

Preferably, aryl substituents and/or heteroaryl substituents (as in R4, R6, R7, R15, R16, R18, R19, R33, R34, R35, R36) contain further substituents such as sulfonic acids or sulfonic acid derivatives and/or alkoxy groups and/or substituted amino groups.

Few examples of simple, non-functionalised compounds of this type are known. In DE 2942931 A1; BASF AG; 7 May 1981; Schmidt R., Koch V. [1] the synthesis of basic structures of formula 5 and their application for dyeing anionically modified textile fibres was described for the first time. No precise information on fluorescence was given.

In Russian Journal of Organic Chemistry, 37(4), 2001, 527-538; Olekhnovich E. P., Boroshko S. L., Korobka I. V., Metelitsa A. V., Olekhnovich L. P. [2] several compounds of this type with a restricted combination of simple, non-functionalised substituents were prepared and studied in more detail with respect to their absorption and emission properties. There, a different synthetic approach was used, which also made the compounds of formulae 1 and 4 accessible. A concrete application of these compounds was not described. These basic structures are only soluble in organic solvents and do not contain any functionalities that cause solubility in aqueous solution and that enable the covalent binding of, for example, biomolecules.

By the preparation of the functionalised bridged benzopyrylium compounds claimed by us, the following advantages are achieved:

By the introduction of linkers and reactive groups to these types of bridged benzopyrylium salts, we enable the formation of a covalent bond with a suitable biomolecule as a prerequisite for use as a fluorescent marker.

Due to the extended choice of substituents, especially by differently substituted alkoxy or amino groups, it is possible to adjust the wavelengths of absorption or emission in a wider range. Surprisingly, the derivatives with R3=NR18R19 are chemically very stable, especially also photostable.

Depending on the basic structure and substitution, the absorption maxima lie in the entire range of visible light, with a focus at 500-530 nm. The compounds show a high Stokes shift in the range of 80 nm. The absorption is measured, for example, at 25° C. in aqueous or ethanolic solutions with an extinction of 1.0 using a Specord 205 from Analytik Jena, wherein the aqueous solutions are preferably phosphate-buffered salt solutions (PBS buffer; approx. 100 mM sodium chloride and 100 mM total phosphate) and have a pH of 7.5. For example, the emission is measured at 25° C. in dilute aqueous or ethanolic solutions with an extinction of 0.1 using a FP-6600 spectrofluorometer from Jasco, wherein the dilute aqueous solutions are preferably phosphate-buffered salt solutions (PBS buffer; about 100 mM sodium chloride and 100 mM total phosphate) and have a pH of 7.5.

Preferably, a compound according to the invention is at least characterised in that the compound is a fluorescent compound.

In one embodiment, the compound according to the invention has an absorption maximum in the wavelength range from 400 nm to 650 nm, preferably from 500 nm to 550 nm.

In one embodiment, the compound according to the invention exhibits an absorption maximum in the wavelength range from 630 nm to 700 nm, preferably from 650 nm to 690 nm.

Preferably, the Stokes shift is at least 40 nm, preferably from 50 nm to 120 nm, more preferably from 70 nm to 90 nm.

Furthermore, because of the higher stability, in one embodiment it is preferred that R3 is NR18R19, wherein R18 and R19 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, more preferably ethyl, (vii) ω-sulfonic acid alkyl (—(CH₂)ₓ—SO₃⁻), wherein x is preferably 1-5, particularly preferably 3, (viii) ω-carboxylic acid alkyl (—(CH₂)_{y}—CO₂H), wherein y is preferably 1-8, particularly preferably 6, and (ix) ethyl esters of (viii), and

wherein NR18R19 is particularly preferably selected from 3-aminopropanesulfonate, N-methylaniline, 4-(methylamino)benzenesulfonate, aniline, 5-carboxypentylamine and 3-carboxypropyl(methyl)amine.

By the substitution with radicals that allow a solubility in water (e.g. sulfonic acids) the applicability of these dyes for analytical or diagnostic purposes in protic solvents is enabled. The hydrophilic character of the different compounds can be adjusted over a certain range by the number of water-solubilising groups. Therefore, a compound according to the invention is preferably water-soluble. Particularly preferably, a compound according to the invention is at least characterised by the fact that at 25° C. at least 1 mg, preferably 2 mg to 4 mg, of the compound are soluble in 1,000 g (1000 mg) of water.

Furthermore, the sulfonic acid groups influence the aggregation behaviour and reduce a non-covalent dimer formation as well as a non-covalent binding to biomolecules and surfaces. In particular, sulfonic acid substituents directly bound to the dye base structure also affect the physicochemical properties of the dyes such that the absorption and emission wavelengths are shifted and, as a rule, a significant increase in quantum yield is achieved.

In certain embodiments, for these reasons, it is preferred that the compound contains at least one sulfonic acid group.

Preferably, a compound according to the invention comprises at least one group selected from a sulfonic acid group, a sulfonic acid derivative, a hydroxy group, an amino group, a carboxylic acid and a carboxylic acid derivative.

Not encompassed by the invention are preferably compounds in which R7 is methoxy and R3 is an amino group, in particular NR18R19, in particular NH(4-HOOCC₆H₄), NH(4-C₂H₅COOC₆H₄), NH(2-C₂H₅COOC₆H₄), NH(C₆H₅), N(C₂H₅)2 or N(CH₂)₂(CH₂)₂O.

Not encompassed by the invention are preferably compounds in which R4 is bromine and R6 and R16 are N(CH₃)₂.

Preferably not encompassed by the invention are compounds in which R6 and R16 are present and one of R6 and R16 is a hydroxy group.

Due to the bridging, benzopyrylium compounds are stably obtained which achieve surprisingly high fluorescence quantum yields in aqueous solution depending on the substituents. Preferably, the fluorescence quantum yield of a compound according to the invention is 0.1 to 0.95, more preferably 0.5 to 0.9. Fluorescence quantum yields are preferably determined at 25° C. in dilute aqueous or ethanolic solutions with an extinction of 0.1 using a Hamamatsu Absolute Photoluminescence Quantum Yield Measurement System C-9920, wherein the dilute aqueous solutions are preferably phosphate-buffered salt solutions (PBS buffer; about 100 mM sodium chloride and 100 mM total phosphate) and have a pH of 7.5. The compounds thus complement the already commercially available dyes with high Stokes Shift or represent better fluorescent alternatives.

Derivatives with R3=OH are present in strongly acidic solutions as hydroxy-benzopyrylium salt and are deprotonated at higher pH values. They then exist as 3-oxo-2H-xanthene with a neutral basic structure. These show a high fluorescence quantum yield and are stable in the physiological pH range.

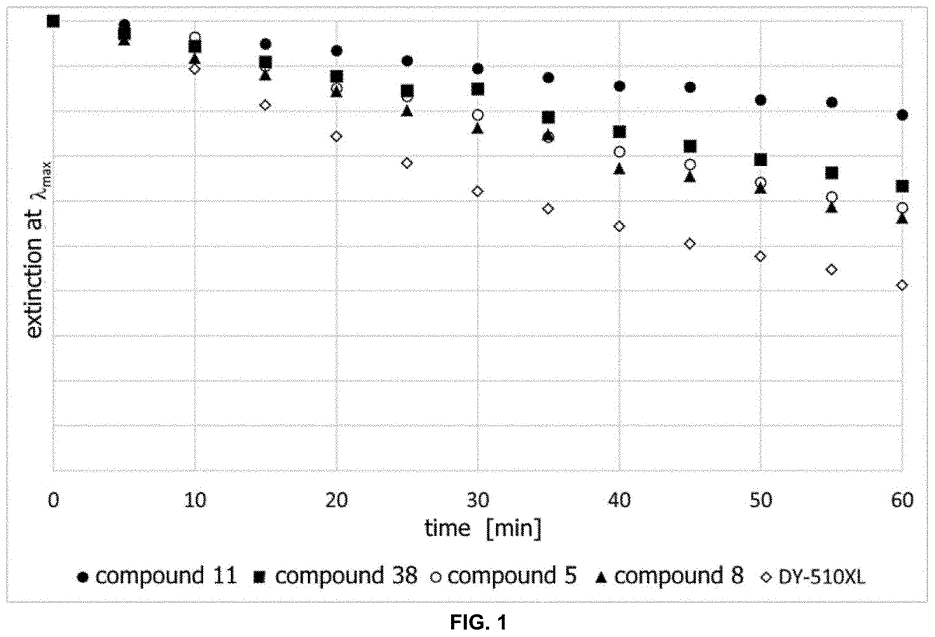

The photostability of the compounds is very good. It is significantly higher than that of comparable coumarin-based dyes, for example than DY-510XL (cf. FIG. 1).

A compound according to the invention is preferably selected from the following compounds and their salts as well as solvates thereof (in the case of ions, the counterion, optionally the counterions, is preferably selected from tetrafluoroborate, chloride and sodium):
6-ethoxy-N,N-diethyl-8,8-dimethyl-7H-xanthene-10-ium-3-amine,
6-[[6-(diethylamino)-1,1-dimethyl-2H-xanthene-10-ium-3-yl]amino]hexanoic acid,
3-[(6-ethoxy-8,8-dimethyl-7H-xanthene-10-ium-3-yl)-(6-ethoxy-6-oxo-hexyl)amino]propane-1-sulfonate,
3-[[6-[5-carboxypentyl(3-sulfonatopropyl)amino]-1,1-dimethyl-2H-xanthene-10-ium-3-yl]amino]propane-1-sulfonate,
3-[[6-[[6-(2,5-dioxopyrrolidin-1-yl)oxy-6-oxo-hexyl]-(3-sulfonatopropyl)amino]-1,1-dimethyl-2H-xanthene-10-ium-3-yl]amino]propane-1-sulfonate,
6-[(6-ethoxy-8,8-dimethyl-7H-xanthene-10-ium-3-yl)-ethyl-amino]hexanoic acid ethyl ester,
3-[[6-[5-carboxypentyl(ethyl)amino]-1,1-dimethyl-2H-xanthene-10-ium-3-yl]amino]propane-1-sulfonate,
3-[5-carboxypentyl-[8,8-dimethyl-6-(N-methylanilino)-7H-xanthene-10-ium-3-yl]amino]propane-1-sulfonate,
4-[[6-[5-carboxypentyl(3-sulfonatopropyl)amino]-1,1-dimethyl-2H-xanthene-10-ium-3-yl]-methyl-amino]benzenesulfonate,
6-[[8,8-dimethyl-6-(N-methylanilino)-7H-xanthene-10-ium-3-yl]-ethyl-amino]hexanoic acid,
4-[[6-[5-carboxypentyl(ethyl)amino]-1,1-dimethyl-2H-xanthene-10-ium-3-yl]-methyl-amino]benzene sulfonate,
6-[(6-anilino-8,8-dimethyl-7H-xanthene-10-ium-3-yl)-ethyl-amino]hexanoic acid,
6-[5-carboxypentyl(ethyl)amino]-1,1-dimethyl-3-(4-sulfonatoanilino)-2H-xanthene-10-ium-4-sulfonate,
6-[[6-[5-carboxypentyl(ethyl)amino]-1,1-dimethyl-2H-xanthene-10-ium-3-yl]amino]naphthalene-2-sulfonate,
6-ethoxy-8,8-dimethyl-7H-xanthene-10-ium-3-ol,
8,8-dimethyl-6-(N-methylanilino)-7H-xanthene-10-ium-3-ol,
6-[[8,8-dimethyl-6-(N-methylanilino)-7H-xanthene-10-ium-3-yl]oxy]hexanoic acid,
3-(5-carboxypentoxy)-8,8-dimethyl-6-(N-methyl-4-sulfonato-anilino)-7H-xanthene-10-ium-2-sulfonate,
3-(9-ethoxy-2,2,4,7,7-pentamethyl-8H-chromeno[3,2-g]quinolin-11-ium-1-yl)propane-1-sulfonate,
6-(9-ethoxy-2,2,4,7,7-pentamethyl-8H-chromeno[3,2-g]quinolin-11-ium-1-yl)hexanoic acid ethyl ester,
3-[9-(5-carboxypentylamino)-2,2,4,7,7-pentamethyl-8H-chromeno[3,2-g]quinolin-11-ium-1-yl]propane-1-sulfonate,
3-[9-[3-carboxypropyl(methyl)amino]-2,2,4,7,7-pentamethyl-8H-chromeno[3,2-g]quinolin-11-ium-1-yl]propane-1-sulfonate,
6-[2,2,4,7,7-pentamethyl-9-(N-methylanilino)-8H-chromeno[3,2-g]quinolin-11-ium-1-yl]hexanoic acid,
4-[[1-(5-carboxypentyl)-2,2,7,7-tetramethyl-4-(sulfonatomethyl)-8H-chromeno[3,2-g]quinolin-11-ium-9-yl]-methyl-amino]benzene sulfonate,
6-[[6-[3-(dimethylamino)anilino]-8,8-dimethyl-7H-xanthene-10-ium-3-yl]-ethyl-amino]hexanoic acid,
6-[[6-(4-aminoanilino)-8,8-dimethyl-7H-xanthene-10-ium-3-yl]-ethyl-amino]hexanoic acid,
6-[[6-[bis(2-pyridylmethyl)amino]-8,8-dimethyl-7H-xanthene-10-ium-3-yl]-ethyl-amino]hexanoic acid,
6-[[8,8-dimethyl-6-[(E)-1H-pyridin-2-ylidenemethyl]-7H-xanthene-10-ium-3-yl]-ethyl-amino]hexanoic acid,
6-[[6-[4-(dimethylamino)phenyl]-8,8-dimethyl-7H-xanthene-10-ium-3-yl]-ethyl-amino]hexanoic acid,
6-[ethyl-(6,8,8-trimethyl-7H-xanthene-10-ium-3-yl)amino]hexanoic acid,
(2E)-1-(5-carboxypentyl)-2-[(E)-3-[6-(diethylamino)-1,1-dimethyl-2H-xanthene-10-ium-3-yl]prop-2-enylidene]-3,3-dimethyl-indoline-5-sulfonate,
3-[(5Z)-3-(5-carboxypentyl)-5-[(2E)-2-[6-(diethylamino)-1,1-dimethyl-2H-xanthene-3-ylidene]ethylidene]-2,4,6-trioxo-hexahydropyrimidine-1-yl]propane-1-sulfonate,
6-(diethylamino)-1,1-dimethyl-2H-xanthene-3-one,
6-[(8,8-dimethyl-6-oxo-7H-xanthene-3-yl)-ethyl-amino]hexanoic acid,
6-[5-carboxypentyl(ethyl)amino]-1,1-dimethyl-3-oxo-2H-xanthene-4-sulfonate,
3-[5-carboxypentyl(8,8-dimethyl-6-oxo-7H-xanthene-3-yl)amino]propane-1-sulfonate,
6-[5-carboxypentyl(3-sulfonatopropyl)amino]-1,1-dimethyl-3-oxo-2H-xanthene-4-sulfonate,
6-hydroxy-1,1-dimethyl-3-oxo-2H-xanthene-4-sulfonate,
6-(5-carboxypentoxy)-1,1-dimethyl-3-oxo-2H-xanthene-4-sulfonate,
6-(2,2,4,7,7-pentamethyl-9-oxo-8H-chromeno[3,2-g]quinolin-1-yl)hexanoic acid,
1-(5-carboxypentyl)-2,2,4,7,7-pentamethyl-9-oxo-8H-chromeno[3,2-g]quinoline sulfonate,
6-[[8,8-dimethyl-6-oxo-5-(4-pyridyl)-7H-xanthene-3-yl]-ethyl-amino]hexanoic acid,
3-[5-Carboxypentyl-[8,8-dimethyl-6-oxo-5-(4-pyridyl)-7H-xanthene-3-yl]amino]propane-1-sulfonate,
2-[3,9-bis(diethylamino)-13,13-dimethyl-chromeno[3,2-b]xanthene-5-ium-14-yl]benzoic acid,
6-[[9-(diethylamino)-13,13-dimethyl-chromeno[3,2-b]xanthene-7-ium-3-yl]-ethyl-amino]hexanoic acid, and
6-[ethyl-(3-methoxy-13,13-dimethyl-chromeno[3,2-b]xanthene-5-ium-9-yl)amino]hexanoic acid.

A compound according to the invention is particularly preferably selected from
6-ethoxy-N,N-diethyl-8,8-dimethyl-7H-xanthene-10-ium-3-amine tetrafluoroborate,
6-[[6-(diethylamino)-1,1-dimethyl-2H-xanthene-10-ium-3-yl]amino]hexanoic acid chloride salt,
3-[(6-ethoxy-8,8-dimethyl-7H-xanthene-10-ium-3-yl)-(6-ethoxy-6-oxo-hexyl)amino]propane-1-sulfonate,
3-[[6-[5-carboxypentyl(3-sulfonatopropyl)amino]-1,1-dimethyl-2H-xanthene-10-ium-3-yl]amino]propane-1-sulfonate sodium salt,
3-[[6-[[6-(2,5-dioxopyrrolidine-1-yl)oxy-6-oxo-hexyl]-(3-sulfonatopropyl)amino]-1,1-dimethyl-2H-xanthene-10-ium-3-yl]amino]propane-1-sulfonate sodium salt,
6-[(6-ethoxy-8,8-dimethyl-7H-xanthene-10-ium-3-yl)-ethyl-amino]hexanoic acid ethyl ester tetrafluoroborate,
3-[[6-[5-carboxypentyl(ethyl)amino]-1,1-dimethyl-2H-xanthene-10-ium-3-yl]amino]propane-1-sulfonate,
3-[5-carboxypentyl-[8,8-dimethyl-6-(N-methylanilino)-7H-xanthene-10-ium-3-yl]amino]propane-1-sulfonate,
4-[[6-[5-carboxypentyl(3-sulfonatopropyl)amino]-1,1-dimethyl-2H-xanthene-10-ium-3-yl]-methyl-amino]benzenesulfonate sodium salt,
6-[[8,8-dimethyl-6-(N-methylanilino)-7H-xanthene-10-ium-3-yl]-ethyl-amino]hexanoic acid chloride salt,
4-[[6-[5-carboxypentyl(ethyl)amino]-1,1-dimethyl-2H-xanthene-10-ium-3-yl]-methyl-amino]benzenesulfonate,
6-[(6-anilino-8,8-dimethyl-7H-xanthene-10-ium-3-yl)-ethyl-amino]hexanoic acid chloride salt,
6-[5-carboxypentyl(ethyl)amino]-1,1-dimethyl-3-(4-sulfonatoanilino)-2H-xanthene-10-ium-4-sulfonate sodium salt,
6-[[6-[5-carboxypentyl(ethyl)amino]-1,1-dimethyl-2H-xanthene-10-ium yl]amino]naphthalene-2-sulfonate,
6-ethoxy-8,8-dimethyl-7H-xanthene-10-ium-3-ol tetrafluoroborate,
8,8-dimethyl-6-(N-methylanilino)-7H-xanthene-10-ium-3-ol tetrafluoroborate,
6-[[8,8-dimethyl-6-(N-methylanilino)-7H-xanthene-10-ium-3-yl]oxy]hexanoic acid chloride salt,
3-(5-carboxypentoxy)-8,8-dimethyl-6-(N-methyl-4-sulfonato-anilino)-7H-xanthene-10-ium-2-sulfonate sodium salt,
3-(9-ethoxy-2,2,4,7,7-pentamethyl-8H-chromeno[3,2-g]quinolin-11-ium-1-yl)propane-1-sulfonate,
6-(9-ethoxy-2,2,4,7,7-pentamethyl-8H-chromeno[3,2-g]quinolin-11-ium-1-yl)hexanoic acid ethyl ester tetrafluoroborate,
3-[9-(5-carboxypentylamino)-2,2,4,7,7-pentamethyl-8H-chromeno[3,2-g]quinolin-11-ium-1-yl]propane-1-sulfonate,
3-[9-[3-carboxypropyl(methyl)amino]-2,2,4,7,7-pentamethyl-8H-chromeno[3,2-g]quinolin-11-ium-1-yl]propane-1-sulfonate,
6-[2,2,4,7,7-pentamethyl-9-(N-methylanilino)-8H-chromeno[3,2-g]quinolin-11-ium-1-yl]hexanoic acid chloride salt,
4-[[1-(5-carboxypentyl)-2,2,7,7-tetramethyl-4-(sulfonatomethyl)-8H-chromeno[3,2-g]quinolin-11-ium-9-yl]-methyl-amino]benzenesulfonate sodium salt,
6-[[6-[3-(dimethylamino)anilino]-8,8-dimethyl-7H-xanthene-10-ium-3-yl]-ethyl-amino]hexanoic acid chloride salt,
6-[[6-(4-aminoanilino)-8,8-dimethyl-7H-xanthene-10-ium-3-yl]-ethyl-amino]hexanoic acid chloride salt,
6-[[6-[bis(2-pyridylmethyl)amino]-8,8-dimethyl-7H-xanthene-10-ium-3-yl]-ethyl-amino]hexanoic acid chloride salt,
6-[[8,8-dimethyl-6-[(E)-1H-pyridin-2-ylidenemethyl]-7H-xanthene-10-ium-3-yl]-ethyl-amino]hexanoic acid chloride salt,
6-[[6-[4-(dimethylamino)phenyl]-8,8-dimethyl-7H-xanthene-10-ium-3-yl]-ethyl-amino]hexanoic acid chloride salt,
6-[ethyl-(6,8,8-trimethyl-7H-xanthene-10-ium-3-yl)amino]hexanoic acid chloride salt,
(2E)-1-(5-Carboxypentyl)-2-[(E)-3-[6-(diethylamino)-1,1-dimethyl-2H-xanthene ium-3-yl]prop-2-enyliden]-3,3-dimethyl-indoline-5-sulfonate,
3-[(5Z)-3-(5-carboxypentyl)-5-[(2E)-2-[6-(diethylamino)-1,1-dimethyl-2H-xanthene-3-ylidene]ethylidene]-2,4,6-trioxo-hexahydropyrimidine-1-yl]propane-1-sulfonate sodium salt,
6-(diethylamino)-1,1-dimethyl-2H-xanthene-3-one,
6-[(8,8-dimethyl-6-oxo-7H-xanthene-3-yl)-ethyl-amino]hexanoic acid,
6-[5-carboxypentyl(ethyl)amino]-1,1-dimethyl-3-oxo-2H-xanthene-4-sulfonate sodium salt,
3-[5-carboxypentyl(8,8-dimethyl-6-oxo-7H-xanthene-3-yl)amino]propane-1-sulfonate sodium salt,
6-[5-carboxypentyl(3-sulfonatopropyl)amino]-1,1-dimethyl-3-oxo-2H-xanthene-4-sulfonate di-sodium salt,
6-hydroxy-1,1-dimethyl-3-oxo-2H-xanthene-4-sulfonate sodium salt,
6-(5-carboxypentoxy)-1,1-dimethyl-3-oxo-2H-xanthene-4-sulfonate sodium salt,
6-(2,2,4,7,7-Pentamethyl-9-oxo-8H-chromeno[3,2-g]quinolin-1-yl)hexanoic acid,
1-(5-carboxypentyl)-2,2,4,7,7-pentamethyl-9-oxo-8H-chromeno[3,2-g]quinoline-10-sulfonate sodium salt,
6-[[8,8-dimethyl-6-oxo-5-(4-pyridyl)-7H-xanthene-3-yl]-ethyl-amino]hexanoic acid,
3-[5-carboxypentyl-[8,8-dimethyl-6-oxo-5-(4-pyridyl)-7H-xanthene-3-yl]amino]propane-1-sulfonate sodium salt,
2-[3,9-bis(diethylamino)-13,13-dimethyl-chromeno[3,2-b]xanthene-5-ium-14-yl]benzoic acid chloride salt,
6-[[9-(diethylamino)-13,13-dimethyl-chromeno[3,2-b]xanthene-7-ium-3-yl]ethyl-amino]hexanoic acid chloride salt, and
6-[ethyl-(3-methoxy-13,13-dimethyl-chromeno[3,2-b]xanthene-5-ium-9-yl)amino]hexanoic acid chloride salt.

A compound according to the invention is particularly preferably selected from the following compounds and salts thereof as well as solvates thereof:
3-[[6-[5-carboxypentyl(3-sulfonatopropyl)amino]-1,1-dimethyl-2H-xanthene-10-ium-3-yl]amino]propane-1-sulfonate,
4-[[6-[5-carboxypentyl(3-sulfonatopropyl)amino]-1,1-dimethyl-2H-xanthene-10-ium-3-yl]-methyl-amino]benzenesulfonate,
6-[5-carboxypentyl(ethyl)amino]-1,1-dimethyl-3-oxo-2H-xanthene-4-sulfonate,
6-[5-carboxypentyl(3-sulfonatopropyl)amino]-1,1-dimethyl-3-oxo-2H-xanthene-4-sulfonate, and
6-(5-carboxypentoxy)-1,1-dimethyl-3-oxo-2H-xanthene-4-sulfonate.

A compound according to the invention is particularly preferably selected from
3-[[6-[5-carboxypentyl(3-sulfonatopropyl)amino]-1,1-dimethyl-2H-xanthene-10-ium-3-yl]amino]propane-1-sulfonate sodium salt,
4-[[6-[5-carboxypentyl(3-sulfonatopropyl)amino]-1,1-dimethyl-2H-xanthene-10-ium-3-yl]-methyl-amino]benzenesulfonate sodium salt,
6-[5-carboxypentyl(ethyl)amino]-1,1-dimethyl-3-oxo-2H-xanthene-4-sulfonate sodium salt,
6-[5-carboxypentyl(3-sulfonatopropyl)amino]-1,1-dimethyl-3-oxo-2H-xanthene-4-sulfonate di-sodium salt, and
6-(5-carboxypentoxy)-1,1-dimethyl-3-oxo-2H-xanthene-4-sulfonate sodium salt.

The compounds according to the invention can be used as dyes for the optical labelling of organic or inorganic recognition units, e.g. of amino acids, peptides, proteins, antibodies, antigens, haptens, enzyme substrates, enzyme cofactors, biotin, carotenoids, hormones, neurohormones, neurotransmitters, growth factors, lectins, toxins, carbohydrates, oligosaccharides, polysaccharides, dextranes, nucleic acids, oligonucleotides, DNA, RNA, biological cells, lipids, receptor-binding pharmaceuticals or organic or inorganic polymeric carriers.

The labelling of the recognition units can be achieved by the formation of ionic or van der Waals interactions between the markers (compounds of the invention) and the materials to be labelled.

Furthermore, it is also possible to covalently link the recognition unit or the carrier material to the fluorophore. This coupling reaction can be carried out in aqueous or predominantly aqueous solution and preferably at room temperature. This produces a fluorescence probe (conjugate) for the qualitative or quantitative determination of different biomaterials or other organic and inorganic materials.

Both the compounds according to the invention and systems derived therefrom can be used in optical, in particular fluorescence-optical, qualitative and quantitative determination methods for the diagnosis of cell properties, in biosensors (point of care measurements), for genome research (DNA sequencing) and in miniaturisation technologies. Typical applications are in cytometry and cell sorting, fluorescence correlation spectroscopy (FCS), ultra-high throughput screening (UHTS), multicolour fluorescence in situ hybridisation (FISH) and microarrays (DNA and protein chips).

A receptor is a molecule that has an affinity for a given ligand. Receptors can be naturally occurring or man-made molecules. Receptors can be used in pure form or bound to other species. Receptors can be bound covalently or non-covalently either directly or through certain coupling mediators to a binding partner.

A ligand is a molecule that is recognised by a particular receptor. Examples of ligands that can be detected by the present invention include, but are not limited to, agonists and antagonists for cell membrane receptors, toxins and other poisons, viral epitopes, hormones such as opiates and steroids, hormone receptors, peptides, enzymes, enzyme substrates, agents acting as cofactors, lectins, sugars, oligonucleotides, nucleic acids, oligosaccharides, proteins and antibodies.

The use of any of the compounds disclosed herein for the purposes and/or methods mentioned herein, in particular as a fluorescent dye and/or in a fluorescent probe or as a fluorescent probe, is also part of the present invention.

The use of one of the compounds disclosed herein as a fluorescent dye and/or in a fluorescent probe or as a fluorescent probe for labelling one or more compounds selected from amino acids, peptides, proteins, antibodies, antigens, haptens, enzyme substrates, enzyme cofactors, biotin, carotenoids, hormones, neurohormones, neurotransmitters, growth factors, lectins, toxins, carbohydrates, oligosaccharides, polysaccharides, dextrans, nucleic acids, oligonucleotides, DNA, RNA, lipids, receptor-binding pharmaceuticals, and cells is also according to the invention.

Compounds according to the invention may have at least one reactive group A in the form of an active ester, the active ester preferably being the same as an NHS ester (N-hydroxysuccinimidyl ester), a sulfo-NHS ester (sulfo-hydroxysuccinimidyl ester), a TFP ester (tetrafluoro-phenyl ester) or an STP ester (p-sulfo-tetrafluoro-phenyl ester), as indicated in the table below.

| A | Active ester |
|---|---|
| 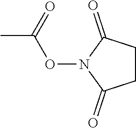 | NHS |
| 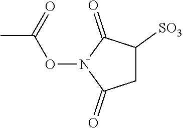 | Sulfo-NHS |
| 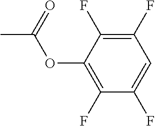 | TFP |
| 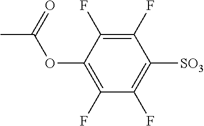 | STP |

In further embodiments, the compounds of the invention may comprise A in the form of a carboxylic acid derivative, wherein the carboxylic acid derivative is preferably a hydrazide, an amine, an iodo-acetamide, a maleimide, an alkyne or an azide, as indicated in the table below.

| A | carboxylic acid amide derivatives |
|---|---|
| 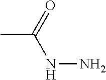 | hydrazide |
| 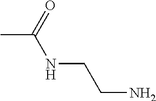 | amine |
| 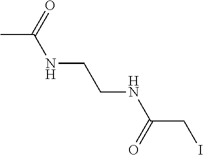 | iodo-acetamide |
| 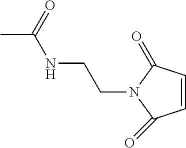 | maleimide |
| 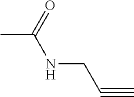 | alkyne |
| 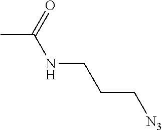 | azide |

Compounds are also described in which A is in the form of a phosphoramidite, where A is a group of the formula

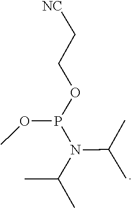

Also subject matter of the invention is a method for the preparation of a compound of formula 1, preferably for the preparation of a compound according to formula 1 in accordance with the invention.

The method comprises the reaction of (E)-(3-ethoxy-5,5-dimethyl-cyclohex-2-en-1-ylidene)-ethyl-oxonium or (E)-[3-ethoxy-5,5-dimethyl-2-(4-pyridyl)cyclohex-2-en-1-ylidene]-ethyl-oxonium with a benzaldehyde compound, wherein the benzaldehyde compound has a benzaldehyde group and the benzaldehyde compound has a hydroxy group in ortho-position to the benzaldehyde group.

Preferably, the reaction is carried out in an organic solvent, wherein the organic solvent is preferably orthoformic acid triethyl ester. Preferably the reaction is carried out at 70° C. to 200° C., more preferably at 90° C. to 130° C. Preferably, (E)-(3-ethoxy-5,5-dimethyl-cyclohex-2-en-1-ylidene)-ethyl-oxonium tetrafluoroborate or (E)-[3-ethoxy-5,5-dimethyl-2-(4-pyridyl)cyclohex-2-en-1-ylidene]-ethyl-oxonium tetrafluoroborate is used. The benzaldehyde compound is preferably selected from 2-hydroxy-4-diethylaminobenzaldehyde, 3-(N-(6-ethoxy-6-oxo-hexyl)-4-formyl hydroxy-anilino)propane-1-sulfonate, ethyl 6-(N-ethyl-4-formyl-3-hydroxy-anilino)hexanoic acid, 2,4-dihydroxybenzaldehyde, 3-(6-formyl-7-hydroxy-2,2,4-trimethyl-1-quinolyl)propane-1-sulfonate and 6-(6-formyl-7-hydroxy-2,2,4-trimethyl-1-quinolyl)hexanoic acid.

The method may comprise, in an upstream reaction, the conversion of the compound 5,5-dimethylcyclohexane-1,3-dione or 3-hydroxy-5,5-dimethyl-2-(4-pyridyl)cyclohex-2-en-1-one using orthoformic acid triethyl ester and tetrafluoroboric acid to the compound (E)-(3-ethoxy-5,5-dimethyl-cyclohex-2-en-1-ylidene)-ethyl-oxonium or (E)-[3-ethoxy-5,5-dimethyl-2-(4-pyridyl)cyclohex-2-en-1-ylidene]-ethyl-oxonium. Preferably, this is carried out at a temperature between 5 and 50° C.

Also a subject matter of the invention is a method for the preparation of a compound of formula 5, preferably for the preparation of a compound of formula 5 according to the invention.

The method comprises reacting a compound of formula 1, preferably 6-ethoxy-N,N-diethyl-8,8-dimethyl-7H-xanthene-10-ium-3-amine or 6-[(6-ethoxy-8,8-dimethyl-7H-xanthene-10-ium-3-yl)-ethyl-amino]hexanoic acid ethyl ester, with a benzaldehyde compound, wherein the benzaldehyde compound has a benzaldehyde group and the benzaldehyde compound has a hydroxy group in ortho-position to the benzaldehyde group.

Preferably, the reaction is carried out in an organic solvent, wherein the organic solvent is preferably glacial acetic acid. Preferably the reaction is carried out at 70° C. to 200° C., more preferably at 90° C. to 110° C. Preferably, 6-ethoxy-N,N-diethyl-8,8-dimethyl-7H-xanthene-10-ium-3-amine tetrafluoroborate or 6-[(6-ethoxy-8,8-dimethyl-7H-xanthene-10-ium-3-yl)-ethyl-amino]hexanoic acid ethyl ester tetrafluoroborate is used. Preferably, the benzaldehyde compound is selected from 2-[4-(diethylamino)-2-hydroxy-benzoyl]benzoic acid, 4-(diethylamino)-2-hydroxy-benzaldehyde and 2-hydroxy-4-methoxy-benzaldehyde.

Preferably, the methods of the invention for preparing a compound of formula 1 or 5 comprise introducing at least one group selected from a sulfonic acid group, a sulfonic acid derivative, an alkoxy group and an amino group, particularly preferably a sulfonic acid group.

The invention is described in more detail below with reference to exemplary embodiments and figures.

They show

FIG. 1: Photostability of compounds 5, 8, 11 and 38 compared to the MegaStokes dye DY-510XL.

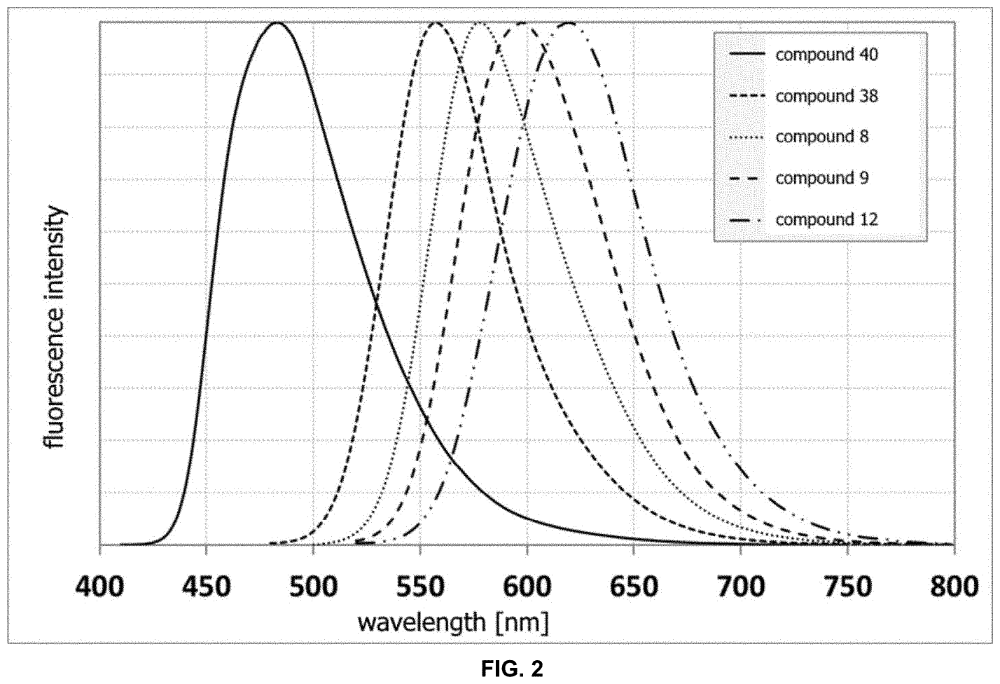

FIG. 2: Emission spectra of selected compounds according to the invention in PBS.

### EXEMPLARY EMBODIMENTS

### Compound 1

### (E)-(3-ethoxy-5,5-dimethyl-cyclohex-2-en-1-ylidene)-ethyl-oxonium tetrafluoroborate

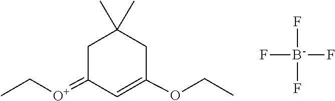

5 mmol of 5,5-dimethylcyclohexane-1,3-dione are suspended in 10 ml of orthoformic acid triethyl ester and 1.5 ml of 48% tetrafluoroboric acid are added at room temperature. After stirring for 30 minutes at RT, 50 ml of dry diethyl ether is added and the mixture is left at RT for several hours. The resulting colourless precipitate is filtered, washed with a little dry ether and dried in vacuo.

Yield: 1.1 g (78%) (C₁₂H₂₁BF₄O₂; 284.10 g/mol)

MS ESI+ (m/z): 197.2 [M⁺]

### Compound 2

### 6-ethoxy-N,N-diethyl-8,8-dimethyl-7H-xanthene-10-ium-3-amine tetrafluoroborate

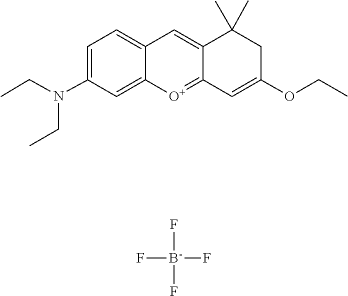 [SMILES: CCOC1=Cc2[o+]c3cc(N(CC)CC)ccc3cc2C(C)(C)C1 ; F[B-](F)(F)F]

3 mmol of compound 1 in 15 ml of orthoformic acid triethyl ester are provided and a solution of 3 mmol of 2-hydroxy-4-diethylaminobenzaldehyde in 10 ml of orthoformic acid triethyl ester are added. The mixture is stirred at 130° C. for 30 minutes. After cooling, it is precipitated with diethyl ether and the precipitate is recrystallized from glacial acetic acid.

Yield: 980 mg (80%) (C₂₁H₂₈BF₄NO₂; 413.26 g/mol)

MS ESI+ (m/z): 326.1 ([M]⁺)

UV-Vis in ethanol: λₘₐₓ: 531 nm; λₑₘ: 605 nm

### Compound 3

### 6-[[6-(diethylamino)-1,1-dimethyl-2H-xanthene-10-ium-3-yl]amino]hexanoic Acid Chloride Salt

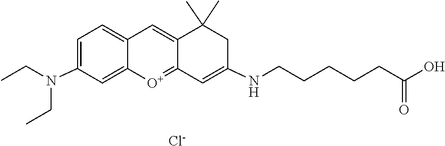 [SMILES: CCN(CC)c1ccc2cc3c([o+]c2c1)C=C(NCCCCCC(=O)O)CC3(C)C ; [Cl-]]

485 μmol compound 2 and 1.5 mmol 6-aminohexanoic acid sodium salt are suspended in 10 ml DMF and stirred at RT for 8 hours. The solvent is distilled off and the residue is purified by RP chromatography.

Yield: 100 mg (46%) (C₂₅H₃₅ClN₂O₃; 447.01 g/mol)

MS ESI− (m/z): 411.2 ([M]⁺)

UV-Vis in PBS: λₘₐₓ: 505 nm; λₑₘ: 570 nm; ε=39,200 l/mol*cm; QY: 0.58

UV-Vis in ethanol: λₘₐₓ: 510 nm; λₑₘ: 575 nm; ε=32,500 l/mol*cm; QY: 0.82

### Compound 4

### 3-[(6-ethoxy-8,8-dimethyl-7H-xanthene-10-ium-3-yl)-(6-ethoxy-6-oxo-hexyl)amino]propane-1-sulfonate

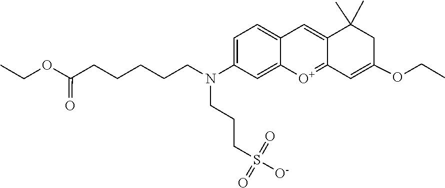 [SMILES: CCOC(=O)CCCCCN(CCCS(=O)(=O)[O-])c1ccc2cc3c([o+]c2c1)C=C(OCC)CC3(C)C]

1 mmol of compound 1 in 5 ml of orthoformic acid triethyl ester are provided and a solution of 1 mmol of the aldehyde 3-(N-(6-ethoxy-6-oxo-hexyl)-4-formyl-3-hydroxy-anilino)propane-1-sulfonate sodium salt in 5 ml of glacial acetic acid are added. The mixture is stirred at 130° C. for 30 minutes. After cooling, it is precipitated with diethyl ether and the precipitate recrystallized from glacial acetic acid.

Yield: 385 mg (72%) (C₂₈H₃₉NO₇S; 533.68 g/mol)

MS ESI+ (m/z): 534.3 ([M+H⁺]⁺)

### Compound 5

### 3-[[6-[5-carboxypentyl(3-sulfonatopropyl)amino]-1,1-dimethyl-2H-xanthene-10-ium-3-yl]amino]propane-1-sulfonate Sodium Salt

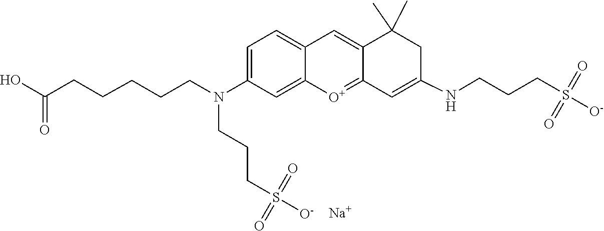 [SMILES: CC1(C)CC(NCCCS(=O)(=O)[O-])=Cc2[o+]c3cc(N(CCCCCC(=O)O)CCCS(=O)(=O)[O-])ccc3cc21 ; [Na+]]

375 μmol compound 4 and 1.5 mmol 3-aminopropane sulfonic acid sodium salt are stirred in 5 ml DMF at 40° C. for 2 hours. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 98 mg (42%) (C₂₇H₃₇N₂NaO₉S₂; 620.71 g/mol)

MS ESI− (m/z): 597.2 (base, [M]⁻); 297.9 (25%, [M−H⁺]²⁻)

UV-Vis in PBS: λₘₐₓ: 505 nm; λₑₘ: 572 nm; ε=45,000 l/mol*cm; QY: 0.72

UV-Vis in ethanol: λₘₐₓ: 512 nm; λₑₘ: 570 nm; ε=49,800 l/mol*cm; QY: 0.87

¹H NMR (400 MHz D₂O): δ (ppm)=1.10 (S, 6H, CH₃); 1.24 (M, 2H, CH₂); 1.40 (M, 2H, CH₂); 1.67 (M, 2H, CH₂); 1.89 (M, 2H, CH₂); 2.04 (M, 2H, CH₂); 2.32 (T, 2H, CH₂); 2.45 (S, 2H, CH₂); 2.87 (T, 2H, CH₂); 2.92 (T, 2H, CH₂); 3.14 (T, 2H, CH₂); 3.35 (T, 2H, CH₂); 3.46 (T, 2H, CH₂); 5.64 (S, 1H, 4-H); 6.16 (S, 1H, 5-H); 6.47 (D, 1H, 7-H); 6.95 (D, 1H, 8-H); 7.26 (S, 1H, 9-H)

¹³C-NMR (400 MHz D₂O): 3 (ppm)=22.27; 23.43; 24.31; 25.71; 26.17; 26.31, 26.86, 33.78; 34.20; 42.14; 42.76; 48.15; 48.29; 49.08; 50.29; 89.31; 95.96; 111.06; 111.34; 126.90; 129.46; 134.58; 151.33; 153.71; 168.62; 169.42; 178.23

### Compound 6

### 3-[[6-[[6-(2,5-dioxopyrrolidine-1-yl)oxy-6-oxo-hexyl]-(3-sulfonatopropyl)amino]-1,1-dimethyl-2H-xanthene-10-ium-3-yl]amino]propane-1-sulfonate Sodium Salt

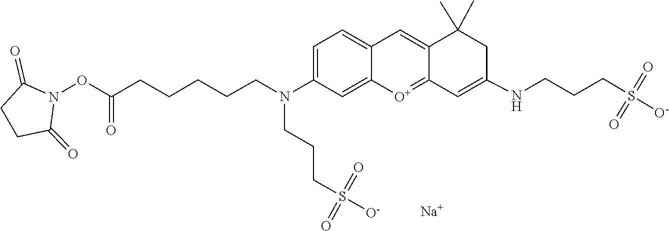 [SMILES: CC1(C)CC(NCCCS(=O)(=O)[O-])=Cc2[o+]c3cc(N(CCCCCC(=O)ON4C(=O)CCC4=O)CCCS(=O)(=O)[O-])ccc3cc21 ; [Na+]]

125 μmol compound 5 are dissolved in 3 ml DMF. At 0° C., 45 mg of TSTU (N,N,N′,N′-tetramethyl-O—(N-succinimidyl)uronium tetrafluoroborate) and 26 μl of DIPEA (diisopropyl-ethylamine) are added and stirred at RT for 20 minutes. The solvent is distilled off in vacuo and the residue is purified by RP chromatography.

Yield: 80 mg (90%) (C₃₁H₄₀N₃NaO₁₁S₂; 717.78 g/mol)

MS ESI− (m/z): 694.2 (base, [M]⁻); 346.5 (25%, [M−H]²⁻)

UV-Vis in ethanol: λₘₐₓ: 512 nm; λₑₘ: 570 nm; ε=44,000 l/mol*cm

### Compound 7

### 6-[(6-ethoxy-8,8-dimethyl-7H-xanthene-10-ium-3-yl)-ethyl-amino]hexanoic acid ethyl ester tetrafluoroborate

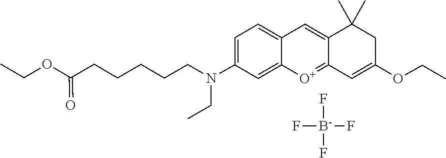 [SMILES: CCOC(=O)CCCCCN(CC)c1ccc2cc3c([o+]c2c1)C=C(OCC)CC3(C)C ; F[B-](F)(F)F]

1 mmol compound 1 and ethyl 6-(N-ethyl-4-formyl-3-hydroxy-anilino)hexanoic acid are reacted according to the synthetic method for compound 2.

Yield: 420 mg (80%) (C₂₇H₃₈BF₄NO₄; 527.40 g/mol)

MS ESI+ (m/z): 440.3 ([M]⁺)

### Compound 8

### 3-[[6-[5-Carboxypentyl(ethyl)amino]-1,1-dimethyl-2H-xanthene-10-ium-3-yl]amino]propane-1-sulfonate

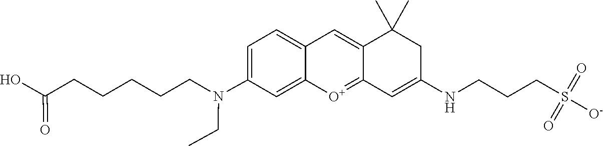 [SMILES: CCN(CCCCCC(=O)O)c1ccc2cc3c([o+]c2c1)C=C(NCCCS(=O)(=O)[O-])CC3(C)C]

375 μmol compound 7 and 3-aminopropane sulfonic acid sodium salt are reacted according to the synthesis method for compound 5.

Yield: 110 mg (58%) (C₂₆H₃₆N₂O₆S; 504.63 g/mol)

MS ESI− (m/z): 503.2 ([M−H⁺]⁻); MS ESI+ (m/z): 505.3 (base, [M+H]⁺); 527.3 (15%, [M+Na]⁺)⁺)

UV-Vis in PBS: λₘₐₓ: 509 nm; λₑₘ: 578 nm; ε=38,500 l/mol*cm; QY: 0.58

UV-Vis in ethanol: λₘₐₓ: 511 nm; λₑₘ: 571 nm; ε=41,600 l/mol*cm; QY 0.84

### Compound 9

### 3-[5-carboxypentyl-[8,8-dimethyl-6-(N-methylanilino)-7H-xanthene-10-ium-3-yl]amino]propane-1-sulfonate

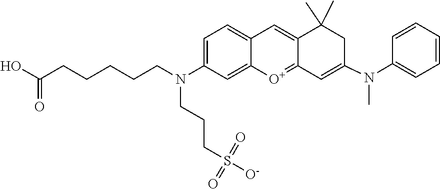 [SMILES: CN(C1=Cc2[o+]c3cc(N(CCCCCC(=O)O)CCCS(=O)(=O)[O-])ccc3cc2C(C)(C)C1)c1ccccc1]

375 μmol compound 4 and 750 μmol N-methylaniline are stirred in 5 ml DMF at 150° C. for 2 hours. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 80 mg (38%) (C₃₁H₃₈N₂O₆S; 566.71 g/mol)

MS ESI− (m/z): 565.2 ([M−H⁺]⁻); MS ESI+ (m/z): 567.3 (80%, [M+H⁺]⁺); 589.3 (base, [M+Na⁺]⁺); 605.3 (70%, [M+Ka⁺]⁺)

UV-Vis in PBS: λₘₐₓ: 523 nm; λₑₘ: 600 nm; ε=39,000 l/mol*cm; QY: 0,24

UV-Vis in ethanol: λₘₐₓ: 531 nm; λₑₘ: 601 nm; ε=41,000 l/mol*cm; QY 0.47

### Compound 10

### 4-[[6-[5-carboxypentyl(3-sulfonatopropyl)amino]-1,1-dimethyl-2H-xanthene-10-ium-3-yl]-methyl-amino]benzenesulfonate sodium Salt

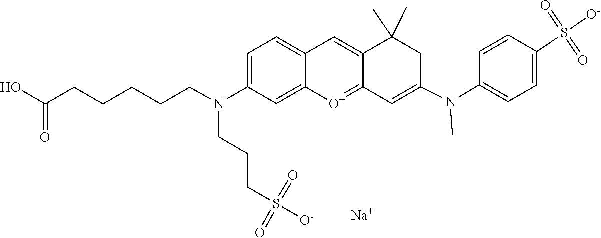 [SMILES: CN(C1=Cc2[o+]c3cc(N(CCCCCC(=O)O)CCCS(=O)(=O)[O-])ccc3cc2C(C)(C)C1)c1ccc(S(=O)(=O)[O-])cc1 ; [Na+]]

177 μmol of compound 9 in 2 ml of oleum (20% SO₃) are dissolved and stirred at RT for 2 hours. The mixture is poured onto ice and stirred for 1 h more at RT. After blunting with sodium carbonate, purification is carried out by RP chromatography.

Yield: 75 mg (64%) (C₃₁H₃₈N₂O₆S; 668.75 g/mol)

MS ESI− (m/z): 645.5 (base, [M]⁻); 322.1 (95%, [M⁻−H]²⁻)

UV-Vis in PBS: λₘₐₓ: 527 nm; λₑₘ: 612 nm; ε=44,000 l/mol*cm; QY: 0.35

UV-Vis in ethanol: λₘₐₓ: 533 nm; λₑₘ: 609 nm; ε=48,000 l/mol*cm; QY 0.57

### Compound 11

### 6-[[8,8-dimethyl-6-(N-methylanilino)-7H-xanthene-10-ium-3-yl]-ethyl-amino]hexanoic Acid Chloride Salt

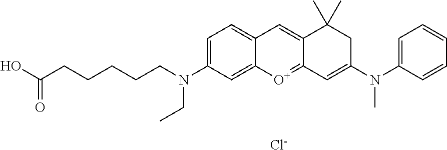 [SMILES: CCN(CCCCCC(=O)O)c1ccc2cc3c([o+]c2c1)C=C(N(C)c1ccccc1)CC3(C)C ; [Cl-]]

375 μmol compound 7 and N-methylaniline are reacted according to the synthesis method for compound 9.

Yield: 100 mg (53%) (C₃₀H₃₇ClN₂O₃; 509.08 g/mol)

MS ESI+ (m/z): 473.3 ([M]⁺); MS ESI− (m/z): 471.2 ([M⁺−2H⁺]⁻)

UV-Vis in PBS: λₘₐₓ: 526 nm; λₑₘ: 606 nm; ε=39,000 l/mol*cm; QY: 0.27

UV-Vis in ethanol: λₘₐₓ: 531 nm; λₑₘ: 601 nm; ε=44,000 l/mol*cm; QY 0.42

### Compound 12

### 4-[[6-[5-carboxypentyl(ethyl)amino]-1,1-dimethyl-2H-xanthene-10-ium-3-yl]-methyl-amino]benzenesulfonate

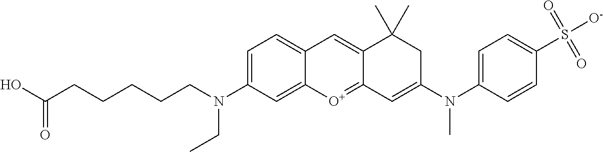 [SMILES: CCN(CCCCCC(=O)O)c1ccc2cc3c([o+]c2c1)C=C(N(C)c1ccc(S(=O)(=O)[O-])cc1)CC3(C)C]

177 μmol of compound 11 are dissolved in 2 ml of oleum (20% SO₃) and stirred at RT for 2 hours. The mixture is poured onto ice and stirred for 1 h more at RT. After blunting with sodium carbonate, purification is carried out by RP chromatography.

Yield: 67 mg (69%) (C₃₀H₃₆N₂O₆S; 552.68 g/mol)

MS ESI− (m/z): 551.2 ([M−H⁻]⁻); MS ESI+ (m/z): 553.4 ([M+H⁺]⁺)

UV-Vis in PBS: λₘₐₓ: 532 nm; λₑₘ: 619 nm; ε=45,000 l/mol*cm; QY: 0.26

UV-Vis in ethanol: λₘₐₓ: 533 nm; λₑₘ: 610 nm; ε=50,000 l/mol*cm; QY 0.51

### Compound 13

### 6-[(6-anilino-8,8-dimethyl-7H-xanthene-10-ium-3-yl)-ethyl-amino]hexanoic Acid Chloride Salt

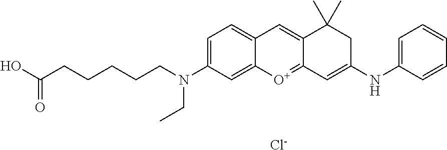 [SMILES: CCN(CCCCCC(=O)O)c1ccc2cc3c([o+]c2c1)C=C(Nc1ccccc1)CC3(C)C ; [Cl-]]

375 μmol compound 7 and 750 μmol dry aniline are stirred in 5 ml DMF at 150° C. for 2 hours. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 50 mg (27%) (C₂₉H₃₅ClN₂O₃; 495.05 g/mol)

MS ESI+ (m/z): 459.3 ([M]⁺); MS ESI− (m/z): 457.2 ([M−2H⁺]⁻)

UV-Vis in PBS: λₘₐₓ: 526 nm; λₑₘ: 609 nm; ε=38,000 l/mol*cm

UV-Vis in ethanol: λₘₐₓ: 532 nm; λₑₘ: 603 nm; ε=44,000 l/mol*cm

### Compound 14

### 6-[5-carboxypentyl(ethyl)amino]-1,1-dimethyl-3-(4-sulfonatoanilino)-2H-xanthene-10-ium-4-sulfonate Sodium Salt

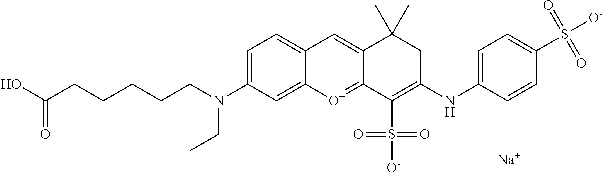 [SMILES: CCN(CCCCCC(=O)O)c1ccc2cc3c([o+]c2c1)C(S(=O)(=O)[O-])=C(Nc1ccc(S(=O)(=O)[O-])cc1)CC3(C)C ; [Na+]]

177 μmol of compound 13 are dissolved in 2 ml of oleum (20% SO₃) and stirred at RT for 2 hours. The mixture is poured onto ice and stirred for 1 h more at RT. After blunting with sodium carbonate, purification is carried out by RP chromatography.

Yield: 30 mg (26%) (C₂₉H₃₃N₂O₉S₂ Na; 640.70 g/mol)

MS ESI− (m/z): 617.3 ([M]⁻)

UV-Vis in PBS: λₘₐₓ: 545 nm; λₑₘ: 625 nm; ε=40,100 l/mol*cm; QY: 0.11

UV-Vis in ethanol: λₘₐₓ: 542 nm; λₑₘ: 616 nm; ε=45,300 l/mol*cm; QY 0.61

### Compound 15

### 6-[[6-[5-carboxypentyl(ethyl)amino]-1,1-dimethyl-2H-xanthene-10-ium-3-yl]amino]naphthalene-2-sulfonate

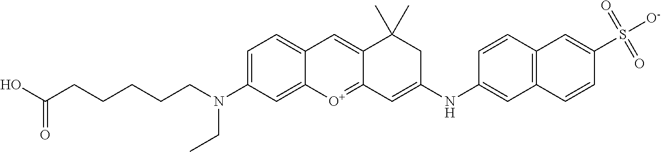 [SMILES: CCN(CCCCCC(=O)O)c1ccc2cc3c([o+]c2c1)C=C(Nc1ccc2cc(S(=O)(=O)[O-])ccc2c1)CC3(C)C]

375 μmol compound 7 and 420 μmol 6-amino-2-naphthalenesulfonic acid hydrate are stirred in 5 ml glacial acetic acid at 120° C. for 8 hours. The solvent is distilled off and 10 ml of 3 M HCl are added to the residue for easter cleavage and boiled at reflux for 1 hour. After neutralisation with sodium hydrogen carbonate, purification is carried out by RP chromatography.

Yield: 18 mg (8%) (C₃₃H₃₆N₂O₆S; 588.71 g/mol)

MS ESI− (m/z): 587.3 ([M−H⁺]⁻)

MS ESI+ (m/z): 589.3 ([M+H⁺]⁺)

UV-Vis in ethanol: λₘₐx: 546 nm; λₑₘ: 619 nm; ε=32,000 l/mol*cm

### Compound 16

### 6-ethoxy-8,8-dimethyl-7H-xanthene-10-ium-3-ol tetrafluoroborate

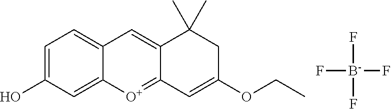 [SMILES: CCOC1=Cc2[o+]c3cc(O)ccc3cc2C(C)(C)C1 ; F[B-](F)(F)F]

5 mmol compound 1 are provided in 25 ml orthoformic acid triethyl ester and 5 mmol 2,4-dihydroxybenzaldehyde are added. The mixture is stirred at 130° C. for 30 minutes. After cooling, it is precipitated with diethyl ether and the precipitate recrystallized from glacial acetic acid.

Yield: 500 mg (28%) (C₁₇H₁₉BF₄O₃; 358.14 g/mol)

MS ESI+ (m/z): 271.2 ([M]⁺)

### Compound 17

### 8,8-dimethyl-6-(N-methylanilino)-7H-xanthene-10-ium-3-ol tetrafluoroborate

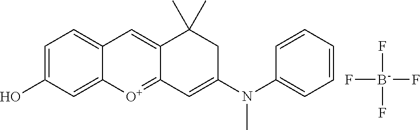 [SMILES: CN(C1=Cc2[o+]c3cc(O)ccc3cc2C(C)(C)C1)c1ccccc1 ; F[B-](F)(F)F]

750 μmol compound 16 and 1.5 mmol N-methylaniline are stirred in 10 ml DMF at 140° C. for 2 hours. After cooling, it is precipitated with diethyl ether and the precipitate recrystallized from glacial acetic acid.

Yield: 125 mg (40%) (C₂₂H₂₂BF₄NO₂; 419.22 g/mol)

MS ESI+ (m/z): 332.3 ([M]⁺)

### Compound 18

### 6-[[8,8-dimethyl-6-(N-methylanilino)-7H-xanthene-10-ium-3-yl]oxy]hexanoic Acid Chloride Salt

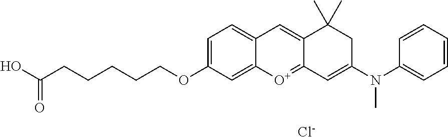 [SMILES: CN(C1=Cc2[o+]c3cc(OCCCCCC(=O)O)ccc3cc2C(C)(C)C1)c1ccccc1 ; [Cl-]]

286 μmol of compound 17 are stirred in 5 ml DMF containing 80 mg K₂CO₃ and 100 mg 6-bromohexanoic acid ethyl ester at 120° C. for 1 hour. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 35 mg (24%) (C₂₈H₃₂ClNO₄; 482.01 g/mol)

MS ESI+ (m/z): 446.4 ([M]⁺)

UV-Vis in ethanol: λₘₐₓ: 452+469 nm; λₑₘ: 520 nm

### Compound 19

### 3-(5-carboxypentoxy)-8,8-dimethyl-6-(N-methyl-4-sulfonato-anilino)-7H-xanthene-10-ium-2-sulfonate Sodium Salt

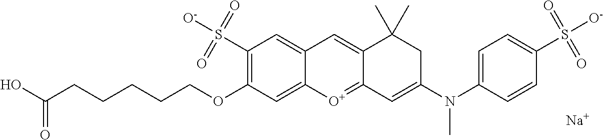 [SMILES: CN(C1=Cc2[o+]c3cc(OCCCCCC(=O)O)c(S(=O)(=O)[O-])cc3cc2C(C)(C)C1)c1ccc(S(=O)(=O)[O-])cc1 ; [Na+]]

35 mg of compound 18 are dissolved in 1 ml of oleum (20% SO₃) and stirred at RT for 2 hours. The mixture is poured onto ice and stirred for 1 h more at RT. After blunting with sodium carbonate, purification is carried out by RP chromatography.

Yield: 27 mg (59%) (C₂₈H₃₀ClNNaO₁₀S₂; 627.66 g/mol)

MS ESI− (m/z): 604.2 ([M]⁻)

UV-Vis in ethanol: λₘₐₓ: 465 nm; λₑₘ: 522 nm; ε=18,000 l/mol*cm.

UV-Vis in PBS: λₘₐₓ: 460 nm; λₑₘ: 509 nm; ε=16.500 l/mol*cm

### Compound 20

### 3-(9-ethoxy-2,2,4,7,7-pentamethyl-8H-chromeno[3,2-g]quinolin-11-ium-1-yl)propane-1-sulfonate

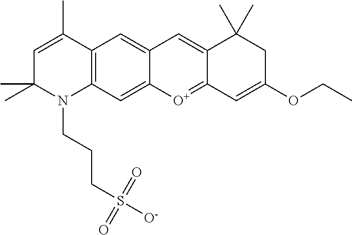 [SMILES: CCOC1=Cc2[o+]c3cc4c(cc3cc2C(C)(C)C1)C(C)=CC(C)(C)N4CCCS(=O)(=O)[O-]]

1 mmol of compound 1 is provided in 5 ml of orthoformic acid triethyl ester and a solution of 1 mmol of the aldehyde 3-(6-formyl-7-hydroxy-2,2,4-trimethyl-1-quinolyl) propane-1-sulfonate sodium salt in 5 ml of glacial acetic acid are added. The mixture is stirred at 130° C. for 30 minutes. After cooling, it is precipitated with diethyl ether and the precipitate recrystallized from glacial acetic acid.

Yield: 320 mg (68%) (C₂₆H₃₃NO₅S; 471.61 g/mol)

MS ESI+ (m/z): 472.2 (base, [M+H⁺]⁺); 494.3 (60%, [M+Na⁺]⁺); 510.3 (20%, [M+K⁺]⁺)

### Compound 21

### 6-(9-ethoxy-2,2,4,7,7-pentamethyl-8H-chromeno[3,2-g]quinolin-11-ium yl)hexanoic Acid Ethyl Ester Tetrafluoroborate

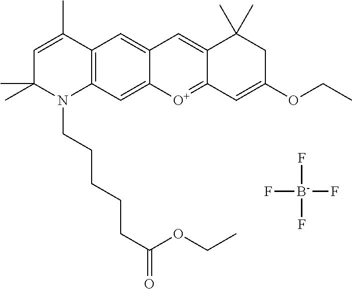 [SMILES: CCOC(=O)CCCCCN1c2cc3[o+]c4c(cc3cc2C(C)=CC1(C)C)C(C)(C)CC(OCC)=C4 ; F[B-](F)(F)F]

1 mmol of compound 1 is provided in 5 ml of orthoformic acid triethyl ester and a solution of 1 mmol of the aldehyde 6-(6-formyl-7-hydroxy-2,2,4-trimethyl-1-quinolyl)hexanoic acid in 5 ml of glacial acetic acid are added. The mixture is stirred at 130° C. for 30 minutes. After cooling, it is precipitated with diethyl ether and the precipitate recrystallized from glacial acetic acid.

Yield: 245 mg (42%) (C₃₁H₄₂BF₄NO₄; 579.47 g/mol)

MS ESI+ (m/z): 492.3 ([M]⁺)

### Compound 22

### 3-[9-(5-carboxypentylamino)-2,2,4,7,7-pentamethyl-8H-chromeno[3,2-g]quinolin-11-ium-1-yl]propane-1-sulfonate

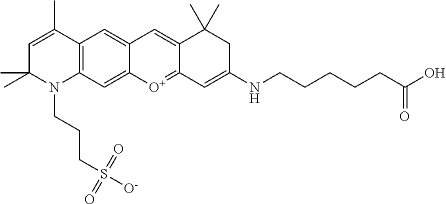 [SMILES: CC1=CC(C)(C)N(CCCS(=O)(=O)[O-])c2cc3[o+]c4c(cc3cc21)C(C)(C)CC(NCCCCCC(=O)O)=C4]

375 μmol compound 20 and 1.5 mmol 6-aminohexanoic acid sodium salt are stirred in 5 ml DMF at 40° C. for 2 hours. The solvent is distilled off and purification is by RP chromatography.

Yield: 25 mg (12%) (C₃₀H₄₀N₂NaO₆S; 556.71 g/mol)

MS ESI− (m/z): 555.2 ([M−H⁺]⁻)

MS ESI+ (m/z): 557.2 (50%, [M+H⁺]⁺); 579.5 (base, [M+Na⁺]⁺); 595.3 (20%, [M+K⁺]⁺

UV-Vis in PBS: λₘₐₓ: 516 nm; λₑₘ: 590 nm; ε=37.500 l/mol*cm

UV-Vis in ethanol: λₘₐₓ: 531 nm; λₑₘ: 589 nm; ε=39,000 l/mol*cm

### Compound 23

### 3-[9-[3-carboxypropyl(methyl)amino]-2,2,4,7,7-pentamethyl-8H-chromeno[3,2-g]quinolin-11-ium-1-yl]propane-1-sulfonate

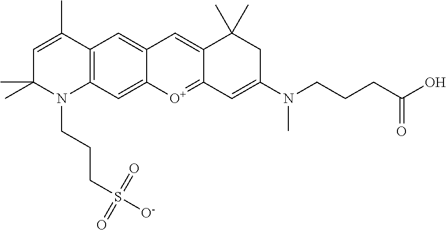 [SMILES: CC1=CC(C)(C)N(CCCS(=O)(=O)[O-])c2cc3[o+]c4c(cc3cc21)C(C)(C)CC(N(C)CCCC(=O)O)=C4]

375 μmol compound 20 and 1.5 mmol N-methylbutyric acid sodium salt are stirred in 5 ml DMF at 40° C. for 2 hours. The solvent is distilled off and purification is by RP chromatography.

Yield: 30 mg (15%) (C₂₉H₃₈N₂O₆S; 542.69 g/mol)

MS ESI− (m/z): 541.2 ([M−H⁺]⁻)

MS ESI+ (m/z): 553.3 (base, [M+H⁺]⁺); 565.5 (20%, [M+Na⁺]⁺); 581.3 (15%, [M+K⁺]⁺

UV-Vis in PBS: λₘₐₓ: 527 nm; λₑₘ: 600 nm; ε=45,400 l/mol*cm; QY: 0.58

UV-Vis in ethanol: λₘₐₓ: 542 nm; λₑₘ: 600 nm; ε=55,000 l/mol*cm; QY: 0.88

### Compound 24

### 6-[2,2,4,7,7-pentamethyl-9-(N-methylanilino)-8H-chromeno[3,2-g]quinolin-11-ium-1-yl]hexanoic Acid Chloride Salt

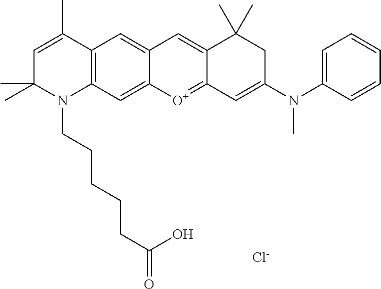 [SMILES: CC1=CC(C)(C)N(CCCCCC(=O)O)c2cc3[o+]c4c(cc3cc21)C(C)(C)CC(N(C)c1ccccc1)=C4 ; [Cl-]]

750 μmol compound 21 and 1.5 mmol N-methylaniline are stirred in 5 ml DMF at 150° C. for 2 hours. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 95 mg (22%) (C₃₄H₄₁NClN₂O₃; 561.15 g/mol)

MS ESI− (m/z): 341.3 (base, [M⁻−H⁺]²⁻); 683.3 (15%, [M]⁻

UV-Vis in PBS: λₘₐₓ: 548 nm; λₑₘ: 632 nm; ε=39.000 l/mol*cm

UV-Vis in ethanol: λₘₐₓ: 555 nm; λₑₘ: 630 nm; ε=41,000 l/mol*cm

### Compound 25

### 4-[[1-(5-carboxypentyl)-2,2,7,7-tetramethyl-4-(sulfonatomethyl)-8H-chromeno[3,2-g]quinolin-11-ium-9-yl]-methyl-amino]benzenesulfonate Sodium Salt

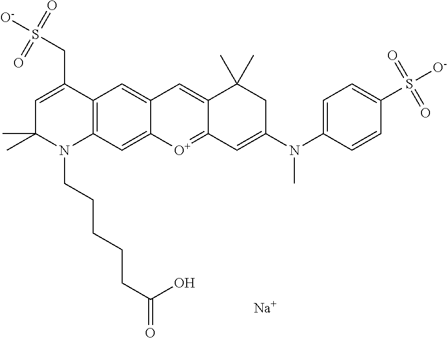 [SMILES: CN(C1=Cc2[o+]c3cc4c(cc3cc2C(C)(C)C1)C(CS(=O)(=O)[O-])=CC(C)(C)N4CCCCCC(=O)O)c1ccc(S(=O)(=O)[O-])cc1 ; [Na+]]

150 μmol of compound 24 are dissolved in 2 ml of oleum (20% SO₃) and stirred at 50° C. for 2 hours. The mixture is poured onto ice and stirred for 1 h more at RT. After blunting with sodium carbonate, purification is carried out by RP chromatography.

Yield: 70 mg (67%) (C₃₄H₃₉NaN₂O₉S₂; 706.80 g/mol)

MS ESI+ (m/z): 525.3 ([M]⁺

UV-Vis in PBS: λₘₐₓ: 540 nm; λₑₘ: 620 nm; ε=42,000 l/mol*cm

UV-Vis in ethanol: λₘₐₓ: 545 nm; λₑₘ: 615 nm; ε=43,000 l/mol*cm

### Compound 26

### 6-[[6-[3-(dimethylamino)anilino]-8,8-dimethyl-7H-xanthene-10-ium-3-yl]-ethyl-amino]hexanoic Acid Chloride Salt

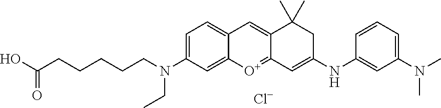 [SMILES: CCN(CCCCCC(=O)O)c1ccc2cc3c([o+]c2c1)C=C(Nc1cccc(N(C)C)c1)CC3(C)C ; [Cl-]]

375 μmol compound 7, 420 μmol 4-amino-N,N-dimethylaniline dihydrochloride and 700 μmol diisopropylethylamine are stirred in 5 ml glacial acetic acid at 120° C. for 2 hours. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 44 mg (22%) (C₃₁H₄₀ClN₃O₃; 538.12 g/mol)

MS ESI+ (m/z): 502.4 ([M]⁺)

UV-Vis in ethanol: λₘₐₓ: 531 nm; λₑₘ: 573 nm; ε=34,000 l/mol*cm

### Compound 27

### 6-[[6-(4-aminoanilino)-8,8-dimethyl-7H-xanthene-10-ium-3-yl]-ethyl-amino]hexanoic Acid Chloride Salt

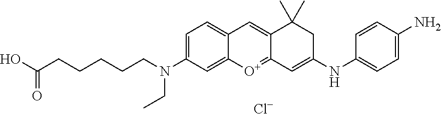 [SMILES: CCN(CCCCCC(=O)O)c1ccc2cc3c([o+]c2c1)C=C(Nc1ccc(N)cc1)CC3(C)C ; [Cl-]]

375 μmol compound 7 and 420 μmol p-phenylenediamine are stirred in 5 ml DMF at 140° C. for 2 hours. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 63 mg (33%) (C₂₉H₃₆ClN₃O₃; 510.07 g/mol)

MS ESI+ (m/z): 474.3 ([M]⁺)

UV-Vis in ethanol: λₘₐₓ: 530 nm; λₑₘ: 612 nm; ε=35,000 l/mol*cm

### Compound 28

### 6-[[6-[bis(2-pyridylmethyl)amino]-8,8-dimethyl-7H-xanthene-10-ium-3-yl]-ethyl-amino]hexanoic Acid Chloride Salt

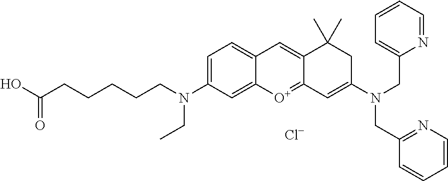 [SMILES: CCN(CCCCCC(=O)O)c1ccc2cc3c([o+]c2c1)C=C(N(Cc1ccccn1)Cc1ccccn1)CC3(C)C ; [Cl-]]

375 μmol compound 7 and 420 μmol di-(2-picolyl)amine are stirred in 5 ml DMF at 140° C. for 2 hours. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 180 mg (80%) (C₃₅H₄₁ClN₄O₃; 601.18 g/mol)

MS ESI+ (m/z): 565.4 ([M]⁺)

UV-Vis in ethanol: λₘₐₓ: 531 nm; λₑₘ: 593 nm; ε=28,000 l/mol*cm

### Compound 29

### 6-[[8,8-dimethyl-6-[(E)-1H-pyridine-2-ylidenemethyl]-7H-xanthene-10-ium-3-yl]-ethyl-amino]hexanoic Acid Chloride Salt

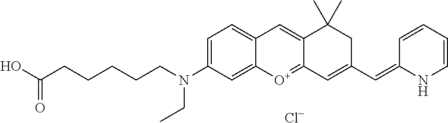 [SMILES: CCN(CCCCCC(=O)O)c1ccc2cc3c([o+]c2c1)C=C(/C=C1\C=CC=CN1)CC3(C)C ; [Cl-]]

1.2 ml of a 1.0 M solution of lithium diisopropylamide in THF/hexane are solved in 10 ml dry THF and 1 mmol dry 2-methylpyridine are added to the mixture at −10° C. This solution is warmed to 0° C. and stirred at 0° C. for 30 minutes. To this is added slowly a solution of 336 μmol of compound 35 in 10 ml of dry THF. When the addition is complete, it is warmed to RT and stirred at RT for 1 hour more. After hydrolysis with dilute HCl, the organic solvents are distilled off and the product is isolated from the aqueous phase by RP chromatography.

Yield: 85 mg (51%) (C₂₉H₃₅ClN₂O₃; 495.05 g/mol)

MS ESI+ (m/z): 459.3 ([M]⁺)

UV-Vis in ethanol: λₘₐₓ: 434 nm; λₑₘ: 541 nm; ε=24,000 l/mol*cm

### Compound 30

### 6-[[6-[4-(dimethylamino)phenyl]-8,8-dimethyl-7H-xanthene-10-ium-3-yl]-ethyl-amino]hexanoic Acid Chloride Salt

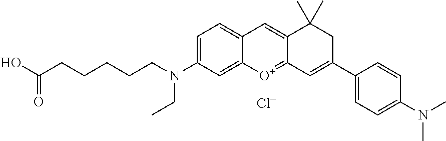 [SMILES: CCN(CCCCCC(=O)O)c1ccc2cc3c([o+]c2c1)C=C(c1ccc(N(C)C)cc1)CC3(C)C ; [Cl-]]

1.0 mol of compound 35 are dissolved in 10 ml of dry pyridine and at −40° C. 5 ml of a 0.5 M solution of 4-(N,N-dimethylaniline)-magnesium bromide in THF are slowly added. After completion of the addition, this solution is warmed to RT and stirred at RT for 3 hours. After hydrolysis with dilute HCl, the organic solvents are distilled off and the product is isolated from the aqueous phase by RP chromatography.

Yield: 100 mg (18%) (C₃₁H₃₉ClN₂O₃; 523.11 g/mol)

MS ESI+ (m/z): 487.5 ([M]⁺)

UV-Vis in ethanol: λₘₐₓ: 664 nm; λₑₘ: 713 nm; ε=30,000 l/mol*cm

### Compound 31

### 6-[ethyl-(6,8,8-trimethyl-7H-xanthene-10-ium-3-yl)amino]hexanoic Acid Chloride Salt

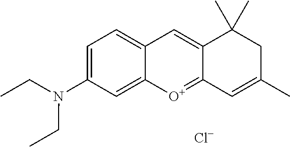 [SMILES: CCN(CC)c1ccc2cc3c([o+]c2c1)C=C(C)CC3(C)C ; [Cl-]]

1.0 mol of compound 35 are dissolved in 10 ml of dry THF and slowly 3 ml of a 1.4 M solution of methyl magnesium bromide in THF/toluene are added while stirring at −20° C. After completion of the addition, this solution is warmed to RT and stirred at RT for 1 hour. After hydrolysis with diluted HCl, the organic solvents are distilled off and the product is isolated from the aqueous phase by RP chromatography.

Yield: 125 mg (30%) (C₂₀H₂₆ClNO; 331.88 g/mol)

MS ESI+ (m/z): 296.1 ([M]⁺)

UV-Vis in ethanol: λₘₐₓ: 538 nm; λₑₘ: 628 nm; ε=9,800 l/mol*cm

### Compound 32

### (2E)-1-(5-sarboxypentyl)-2-[(E)-3-[6-(diethylamino)-1,1-dimethyl-2H-xanthene ium-3-yl]prop-2-enyliden]-3,3-dimethyl-indoline-5-sulfonate

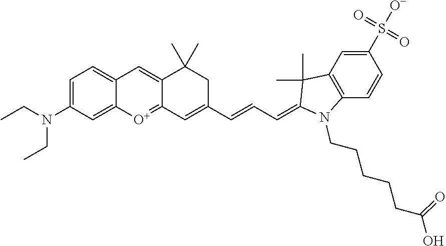 [SMILES: CCN(CC)c1ccc2cc3c([o+]c2c1)C=C(/C=C/C=C1/N(CCCCCC(=O)O)c2ccc(S(=O)(=O)[O-])cc2C1(C)C)CC3(C)C]

1.0 mol of compound 31 and 1.1 mmol of 2-[(E)-2-anilinovinyl]-1-(5-carboxypentyl)-3,3-dimethyl-indole-1-ium-5-sulfonate are dissolved in 4 ml of acetic anhydride and 4 ml of glacial acetic acid and boiled at reflux for 15 minutes with the addition of 250 mg of sodium acetate. After cooling, an oily precipitate is obtained by precipitation with diethyl ether, which precipitate is purified by RP chromatography.

Yield: 92 mg (14%) (C₃₈H₄₆N₂O₆S; 658.85 g/mol)

MS ESI+ (m/z): 659.4 (base, [M+H⁺]⁺); 681.6 (30%, [M+Na⁺]⁺); 697.6 (20%, [M+K⁺]⁺

MS ESI− (m/z): 657.3 ([M−H⁺]⁻)

UV-Vis in PBS: λₘₐₓ: 755 nm; λₑₘ: 788 nm; ε=75.000 l/mol*cm

UV-Vis in ethanol: λₘₐₓ: 770 nm; λₑₘ: 795 nm; ε=143,600 l/mol*cm

### Compound 33

### 3-[(5Z)-3-(5-carboxypentyl)-5-[(2E)-2-[6-(diethylamino)-1,1-dimethyl-2H-xanthene-3-ylidene]ethylidene]-2,4,6-trioxo-hexahydropyrimidin-1-yl]propane-1-sulfonate Sodium Salt

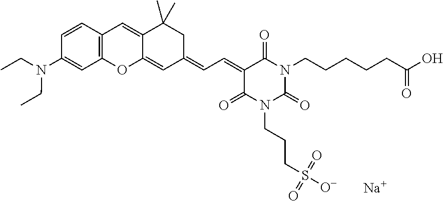 [SMILES: CCN(CC)c1ccc2c(c1)OC1=C/C(=C/C=C3/C(=O)N(CCCCCC(=O)O)C(=O)N(CCCS(=O)(=O)[O-])C3=O)CC(C)(C)C1=C2 ; [Na+]]

1.0 mol of compound 31 and 1 mmol of 3-[5-formyl-3-(6-methoxy-6-oxo-hexyl)-2,4,6-trioxo-hexahydropyrimidine-1-yl]propane-1-sulfonate sodium salt are dissolved in 5 ml of acetic anhydride and 5 ml of glacial acetic acid and boiled at reflux for 15 minutes with the addition of 250 mg of sodium acetate. After cooling, a precipitate is obtained by precipitation with diethyl ether. 10 ml 3 M HCl and 10 ml acetone are added to the precipitate for ester cleavage and boiled at reflux for 1 hour. After neutralisation with sodium hydrogen carbonate, purification is carried out by RP chromatography.

Yield: 150 mg (22%) (C₃₄H₄₂NaN₃O₉S; 691.77 g/mol)

MS ESI− (m/z): 668.1 ([M]⁻)

UV-Vis in ethanol: λₘₐₓ: 683 nm; λₑₘ: 719 nm; ε=67,000 l/mol*cm

### Compound 34

### 6-(diethylamino)-1,1-dimethyl-2H-xanthene-3-one

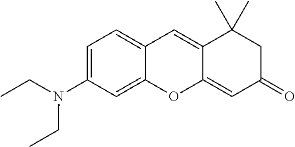 [SMILES: CCN(CC)c1ccc2c(c1)OC1=CC(=O)CC(C)(C)C1=C2]

500 μmol compound 2 are stirred in 50 ml acetone and 10 ml 0.5 M pH9 buffer at 50° C. for 1 hour. The solvent is distilled off and purification is carried out by RP chromatography.

Yield: 80 mg (54%) red-brown oil (C₁₉H₂₃NO₂; 297.39 g/mol)

MS ESI− (m/z): 298.1 (base, [M+H]⁺); 617.4 (90%, [2M+Na])⁺

UV-Vis in PBS: λₘₐₓ: 450 nm; λₑₘ: 558 nm; ε=23.00 l/mol*cm

UV-Vis in water pH 3: λₘₐₓ: x nm; λₑₘ: x nm (present as hydroxy-BPS)

UV-Vis in ethanol: λₘₐₓ: 440 nm; λₑₘ: 540 nm; ε=24,800 l/mol*cm

### Compound 35

### 6-[(8,8-dimethyl-6-oxo-7H-xanthene-3-yl)-ethyl-amino]hexanoic Acid

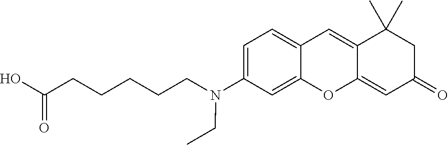 [SMILES: CCN(CCCCCC(=O)O)c1ccc2c(c1)OC1=CC(=O)CC(C)(C)C1=C2]

500 μmol compound 7 are stirred in 50 ml acetone and 10 ml 0.5 M pH9 buffer at 50° C. for 1 hour. The solvent is distilled off and purification is carried out by RP chromatography.

Yield: 150 mg (78%) (C₂₃H₂₉NO₄; 383.48 g/mol)

MS ESI− (m/z): 382.2 ([M−H⁺]⁻); MS ESI+ (m/z): 384.3 ([M+H⁺]⁺)

UV-Vis in PBS: λₘₐₓ: 457 nm; λₑₘ: 559 nm; ε=24,200 l/mol*cm

UV-Vis in ethanol: λₘₐₓ: 441 nm; λₑₘ: 540 nm; ε=25,300 l/mol*cm

### Compound 36

### 6-[5-carboxypentyl(ethyl)amino]-1,1-dimethyl-3-oxo-2H-xanthene-4-sulfonate Sodium Salt

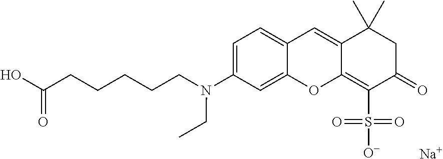 [SMILES: CCN(CCCCCC(=O)O)c1ccc2c(c1)OC1=C(S(=O)(=O)[O-])C(=O)CC(C)(C)C1=C2 ; [Na+]]

177 μmol of compound 35 are dissolved in 2 ml of oleum (20% SO₃) and stirred at RT for 2 hours. The mixture is poured onto ice and stirred for 1 h more at RT. After blunting with sodium carbonate, purification is carried out by RP chromatography.

Yield: 17 mg (20%) (C₂₃H₂₈NO₇S Na; 485.53 g/mol)

MS ESI− (m/z): 462.0 (60%, [M]⁻); 230.4 (base, [M−H⁺]²⁻)

UV-Vis in PBS: λₘₐₓ: 482 nm; λₑₘ: 565 nm; ε=30,100 l/mol*cm; QY: 0.66

UV-Vis in ethanol: λₘₐₓ: 446 nm; λₑₘ: 542 nm; ε=25,000 l/mol*cm; QY 0.90

### Compound 37

### 3-[5-carboxypentyl-(8,8-dimethyl-6-oxo-7H-xanthene-3-yl)amino]propane-1-sulfonate Sodium Salt

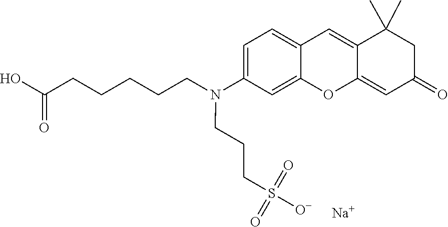 [SMILES: CC1(C)CC(=O)C=C2Oc3cc(N(CCCCCC(=O)O)CCCS(=O)(=O)[O-])ccc3C=C21 ; [Na+]]

375 μmol compound 4 are stirred in 50 ml acetone and 10 ml 0.5 M pH9 buffer at 50° C. for 1 hour. The solvent is distilled off and the residue is purified by RP chromatography with acetonitrile-water gradient.

Yield: 105 mg (56%) (C₂₄H₃₀NO₇S Na; 499.55 g/mol)

MS ESI− (m/z): 476.0 (base, [M]⁻); 237.5 (15%, [M⁻−H⁺]²⁻)

UV-Vis in PBS: λₘₐₓ: 454 nm; λₑₘ: 553 nm; ε=26,000 l/mol*cm

UV-Vis in ethanol: λₘₐₓ: 439 nm; λₑₘ: 538 nm; ε=27,000 l/mol*cm

### Compound 38

### 6-[5-carboxypentyl(3-sulfonatopropyl)amino]-1,1-dimethyl-3-oxo-2H-xanthene-4-sulfonate di-sodium SALT

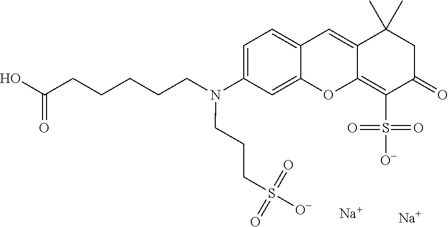 [SMILES: CC1(C)CC(=O)C(S(=O)(=O)[O-])=C2Oc3cc(N(CCCCCC(=O)O)CCCS(=O)(=O)[O-])ccc3C=C21 ; [Na+] ; [Na+]]

177 μmol of compound 37 are dissolved in 2 ml of oleum (20% SO₃) and stirred at RT for 2 hours. The mixture is poured onto ice and stirred for 1 h more at RT. After blunting with sodium carbonate, purification is carried out by RP chromatography.

Yield: 90 mg (85%) (C₂₄H₂₉NO₁₀S Na; 601.60 g/mol)

MS ESI− (m/z): 556.2 (15%, [M²⁻+H⁺]⁻); 277.6 (base, [M]²⁻)

UV-Vis in PBS: λₘₐₓ: 479 nm; λₑₘ: 557 nm; ε=28,000 l/mol*cm; QY: 0.78

UV-Vis in ethanol: λₘₐₓ: 463 nm; λₑₘ: 544 nm; ε=31,000 l/mol*cm; QY 0.91

¹H NMR (400 MHz D₂O): δ (ppm)=1.26 (S, 6H, CH₃); 1.32 (M, 2H, CH₂); 1.51 (M, 2H, CH₂); 1.54 (M, 2H, CH₂); 1.94 (M, 2H, CH₂); 2.30 (T, 2H, CH₂); 2.31 (S, 2H, CH₂); 2.89 (T, 2H, CH₂); 3.27 (T, 2H, CH₂); 3.43 (T, 2H, CH₂); 6.71 (D, 1H, 7-H); 6.86 (S, 1H, 5-H); 7.34 (D, 1H, 8-H); 7.52 (S, 1H, 9-H)

¹³C-NMR (400 MHz D₂O): δ (ppm)=22.02; 24.23; 25.61; 25.98; 26.32; 33.34; 33.91; 48.47; 49.29; 50.23; 50.57; 97.71; 110.71; 111.46; 112.52; 128.02; 129.20; 133.93; 150.95; 153.95; 165.49; 179.17; 193.27

### Compound 39

### 6-hydroxy-1,1-dimethyl-3-oxo-2H-xanthene-4-sulfonate Sodium Salt

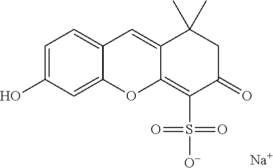 [SMILES: CC1(C)CC(=O)C(S(=O)(=O)[O-])=C2Oc3cc(O)ccc3C=C21 ; [Na+]]

750 μmol of compound 16 are stirred in 50 ml acetone and 10 ml 0.5 M pH9 buffer at 50° C. for 1 hour. The solvent is distilled off and purification is carried out by RP chromatography.

Yield: 115 mg (45%) (C₁₅H₁₃NaO₆S; 344.31 g/mol)

MS ESI− (m/z): 321.3 (base, [M]⁻)

UV-Vis in PBS: λₘₐₓ: 409 nm; λₑₘ: 485 nm

UV-Vis in buffer pH 9: λₘₐₓ: 462 nm; λₑₘ: 536 nm

### Compound 40

### 6-(5-carboxypentoxy)-1,1-dimethyl-3-oxo-2H-xanthene-4-sulfonate Sodium Salt

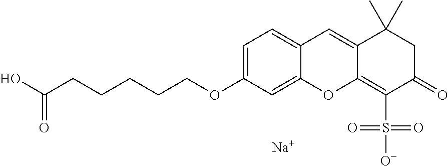 [SMILES: CC1(C)CC(=O)C(S(=O)(=O)[O-])=C2Oc3cc(OCCCCCC(=O)O)ccc3C=C21 ; [Na+]]

290 μmol of compound 39 are stirred in 5 ml DMF with 80 mg K₂CO₃ and 100 mg 6-bromohexanoic acid ethyl ester at 120° C. for 1 hour. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 33 mg (25%) (C₂₁H₂₃NaO₈S Na; 458.46 g/mol)

MS ESI− (m/z): 435.2 (base, [M]⁻); 217.0 (30%, [M⁻−H⁺]²⁻)

UV-Vis in PBS: λₘₐₓ: 407 nm; λₑₘ: 483 nm; ε=21,000 l/mol*cm

UV-Vis in ethanol: λₘₐₓ: 403 nm; λₑₘ: 487 nm; ε=19,500 l/mol*cm

### Compound 41

### 6-(2,2,4,7,7-pentamethyl-9-oxo-8H-chromeno[3,2-g]quinolin-1-yl)hexanoic Acid

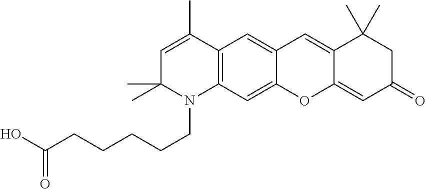 [SMILES: CC1=CC(C)(C)N(CCCCCC(=O)O)c2cc3c(cc21)C=C1C(=CC(=O)CC1(C)C)O3]

375 μmol compound 21 are stirred in 50 ml acetone and 10 ml 0.5 M pH9 buffer at 50° C. for 1 hour. The solvent is distilled off. To the residue 10 ml 3 M HCl is added for ester cleavage and boiled at reflux for 1 hour. After neutralisation with sodium hydrogen carbonate, purification is carried out by RP chromatography.

purified by RP chromatography with acetonitrile-water gradient.

Yield: 60 mg (38%) (C₂₇H₃₃NO₄; 435.56 g/mol)

MS ESI+ (m/z): 436.2 ([M+H⁺]⁺)

UV-Vis in PBS: λₘₐₓ: 460 nm; λₑₘ: 575 nm; ε=23,000 l/mol*cm

UV-Vis in ethanol: λₘₐₓ: 463 nm; λₑₘ: 557 nm; ε=24,000 l/mol*cm

### Compound 42

### 1-(5-carboxypentyl)-2,2,4,7,7-pentamethyl-9-oxo-8H-chromeno[3,2-g]quinoline-10-sulfonate Sodium Salt

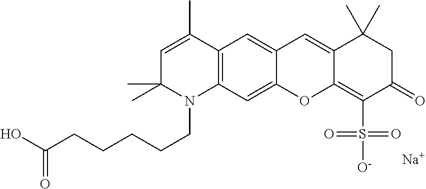 [SMILES: CC1=CC(C)(C)N(CCCCCC(=O)O)c2cc3c(cc21)C=C1C(=C(S(=O)(=O)[O-])C(=O)CC1(C)C)O3 ; [Na+]]

177 μmol of compound 41 are dissolved in 2 ml of oleum (20% SO₃) and stirred at RT for 2 hours. The mixture is poured onto ice and stirred for 1 h more at RT. After blunting with sodium carbonate, purification is carried out by RP chromatography.

Yield: 45 mg (48%) (C₂₇H₃₂NO₇S Na; 537.60 g/mol)

MS ESI− (m/z): 514.2 ([M]⁻)

UV-Vis in PBS: λₘₐₓ: 487 nm; λₑₘ: 578 nm; ε=28,000 l/mol*cm

UV-Vis in ethanol: λₘₐₓ: 473 nm; λₑₘ: 559 nm; ε=30,000 l/mol*cm

### Compound 43

### (E)-[3-ethoxy-5,5-dimethyl-2-(4-pyridyl)cyclohex-2-en-1-ylidene]-ethyl-oxonium tetrafluoroborate

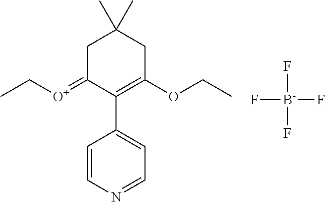 [SMILES: CCOC1=C(c2ccncc2)/C(=[O+]/CC)CC(C)(C)C1 ; F[B-](F)(F)F]

4 mmol of 3-hydroxy-5,5-dimethyl-2-(4-pyridyl)cyclohex-2-en-1-one are suspended in 10 ml of orthoformic acid triethyl ester and 1.2 ml of 48% tetrafluoroboric acid are added at room temperature. After stirring for 30 minutes at RT, 50 ml of dry diethyl ether are added and the mixture is left at RT for several hours. The resulting slightly yellowish precipitate is filtered, washed with a little dry ether and dried in vacuo.

Yield: 970 mg (67%) (C₁₇H₂₄BF₄NO₂; 361.18 g/mol)

MS ESI+ (m/z): 274.2 [M⁺]

### Compound 44

### 6-[[8,8-dimethyl-6-oxo-5-(4-pyridyl)-7H-xanthene-3-yl]-ethyl-amino]hexanoic Acid

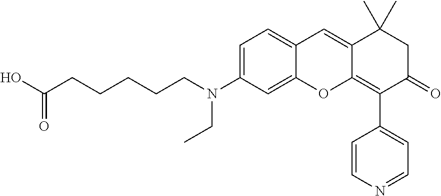 [SMILES: CCN(CCCCCC(=O)O)c1ccc2c(c1)OC1=C(c3ccncc3)C(=O)CC(C)(C)C1=C2]

1 mmol of compound 43 and 1 mmol of ethyl 6-(N-ethyl-4-formyl-3-hydroxy-anilino)hexanoic acid are reacted according to the synthetic method for compound 2. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 97 mg (21%) (C₂₈H₃₂N₂O₄; 460.56 g/mol)

MS ESI+ (m/z): 460.3 ([M+H⁺]⁺)

UV-Vis in PBS: λₘₐₓ: 472 nm; λₑₘ: 573 nm; ε=13,000 l/mol*cm

UV-Vis in buffer pH 5: λₘₐₓ: 523 nm; λₑₘ: 600 nm; ε=13.300 l/mol*cm

### Compound 45

### 3-[5-carboxypentyl-[8,8-dimethyl-6-oxo-5-(4-pyridyl)-7H-xanthene-3-yl]amino]propane-1-sulfonate Sodium Salt

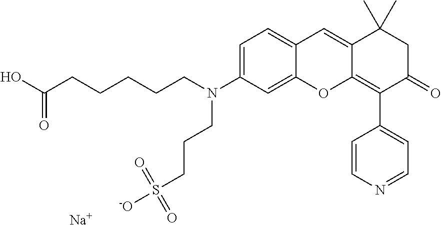

1 mmol of compound 43 and 1 mmol of ethyl 6-(N-ethyl-4-formyl-3-hydroxy-anilino)hexanoic acid are reacted according to the synthetic method for compound 2. The solvent is distilled off and 10 ml of 3 M HCl are added to the residue for ester cleavage and boiled at reflux for 1 hour. After neutralisation with sodium hydrogen carbonate, purification is carried out by RP chromatography.

Yield: 97 mg (21%) (C₂₉H₃₄N₂O₄S; 554.65 g/mol)

MS ESI− (m/z): 553.2 ([M−H⁺]⁻)

MS ESI+ (m/z): 555.4 ([M+H⁺]⁺)

UV-Vis in PBS: λₘₐₓ: 470 nm; λₑₘ: 570 nm; ε=15.000 l/mol*cm

UV-Vis in buffer pH 5: λₘₐₓ: 524 nm; λₑₘ: 594 nm; ε=15.400 l/mol*cm

### Compound 46

### 2-[3,9-bis(diethylamino)-13,13-dimethyl-chromeno[3,2-b]xanthene-5-ium-14-yl]benzoic Acid Chloride Salt

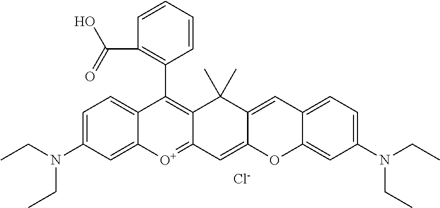 [SMILES: CCN(CC)c1ccc2c(c1)OC1=Cc3[o+]c4cc(N(CC)CC)ccc4c(-c4ccccc4C(=O)O)c3C(C)(C)C1=C2 ; [Cl-]]

1 mmol of compound 2 and 1 mmol of 2-[4-(diethylamino)-2-hydroxy-benzoyl] benzoic acid are stirred in 5 ml of glacial acetic acid at 110° C. for 16 hours. After cooling, it is precipitated with diethyl ether and the precipitate purified by RP chromatography.

Yield: 214 mg (34%) (C₃₇H₃₉ClN₂O₄; 611.17 g/mol)

MS ESI+ (m/z): 575.4 ([M]⁺)

UV-Vis in ethanol: λₘₐₓ: 664 nm; λₑₘ: 710 nm; ε=60,000 l/mol*cm

UV-Vis in PBS: λₘₐₓ: 663 nm; λₑₘ: 711 nm; ε=42.000 l/mol*cm

### Compound 47

### 6-[[9-(diethylamino)-13,13-dimethyl-chromeno[3,2-b]xanthene-7-ium-3-yl]-ethyl-amino]hexanoic Acid Chloride Salt

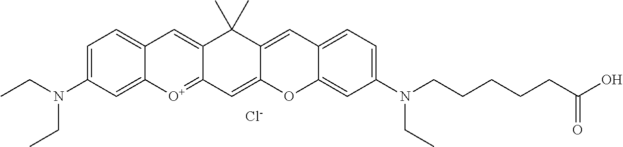 [SMILES: CCN(CC)c1ccc2cc3c([o+]c2c1)C=C1Oc2cc(N(CC)CCCCCC(=O)O)ccc2C=C1C3(C)C ; [Cl-]]

500 μmol compound 7 and 500 μmol 4-(diethylamino)-2-hydroxy-benzaldehyde are stirred in 5 ml glacial acetic acid at 110° C. for 16 hours. After cooling, it is precipitated with diethyl ether and the precipitate purified by RP chromatography.

Yield: 70 mg (24%) (C₃₄H₄₁ClN₂O₄; 577.15 g/mol)

MS ESI+ (m/z): 541.3 ([M]⁺)

UV-Vis in ethanol: λₘₐₓ: 679 nm; 713 nm; ε=80,300 l/mol*cm

UV-Vis in PBS: λₘₐₓ: 670 nm; 710 nm; ε=37.000 l/mol*cm

### Compound 48

### 6-[ethyl-(3-methoxy-13,13-dimethyl-chromeno[3,2-b]xanthene-5-ium-9-yl)amino]hexanoic Acid Chloride Salt

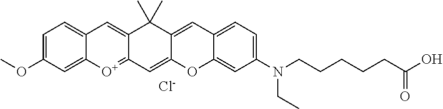 [SMILES: CCN(CCCCCC(=O)O)c1ccc2c(c1)OC1=Cc3[o+]c4cc(OC)ccc4cc3C(C)(C)C1=C2 ; [Cl-]]

1 mmol compound 7 and 1 mmol 2-hydroxy-4-methoxy-benzaldehyde are stirred in 5 ml glacial acetic acid at 110° C. for 16 hours. After cooling, it is precipitated with diethyl ether and the precipitate purified by RP chromatography.

Yield: 43 mg (8%) (C₃₁H₃₄ClNO₅; 536.06 g/mol)

MS ESI+ (m/z): 500.2 ([M]⁺)

UV-Vis in ethanol: λₘₐₓ: 654 nm; 712 nm; ε=53,000 l/mol*cm.

Photostability of Selected Compounds According to the Invention

FIG. 1 shows the results of the irradiation of aqueous solutions (PBS pH 7.5, 100 mM & 100 mM NaCl plus 5 mM NaN₃) of compounds 5, 8, 11 and 38 in comparison to the MegaStokes dye DY-510XL. The solutions were adjusted to an extinction of “1” in the absorption maximum at a layer thickness of 1 cm and irradiated with white light from the 150 W Xe lamp of a fluorescence spectrometer (JASCO FP-6600, monochromator at 0 nm, slit position L: 10 nm) and the absorption in the maximum was monitored in 5 min intervals over one hour.

Fluorescence Maxima of Selected Compounds According to the Invention

FIG. 2 shows the fluorescence maxima of selected compounds.

The invention relates to novel, water-soluble fluorescent dyes with high fluorescence quantum yields based on oxygen-containing heterocycles, their reactive derivatives and dye conjugates, and their use for labelling samples and detecting analytes. The compounds of the new dye class are compatible with commercial excitation light sources and are characterised by Stokes shifts of more than 50 nm.

### FURTHER ASPECTS OF THE INVENTION

Further preferred aspects of the invention are set out below:

### 1. Aspect

A compound of the general formula 1

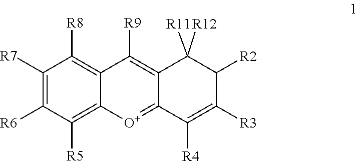

and salts and solvates thereof, wherein
R11 and R12 are each independently of the other hydrogen or alkyl,
R2 is hydrogen, alkyl or alkenyl,
R3 is hydrogen, alkyl, aryl, hydroxy, alkoxy, aryloxy, NR18R19 or a group Q,
wherein R18 and R19 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, more preferably ethyl, (vii) ω-sulfonic acid alkyl (—CH₂)ₓ—SO₃⁻) where x is 1-5, (viii) ω-carboxylic acid alkyl (—(CH₂)_{y}—CO₂H) where y is 1-8, and (ix) ethyl esters of (viii),
and Q is a heterocyclic structure selected from a structure of formula 2 or 3

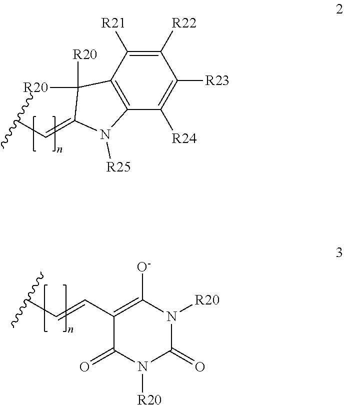

where n=1, 2 or 3; wherein each R20 is independently of the other alkyl, ω-sulfonic acid alkyl (—(CH₂)ₓ—SO₃⁻) or a reactive group A bound through a linker L, each R21, R22, R23, R24 is independently of the other hydrogen, a sulfonic acid or a sulfonic acid derivative, R25 is hydrogen, alkyl, ω-sulfonic acid alkyl (—(CH₂)ₓ—SO₃⁻) or a reactive group A bound through a linker L, wherein each x is an integer from 1-5, and
R4 is hydrogen, bromine, chlorine, sulfonic acid or a sulfonic acid derivative, alkyl, aryl or heteroaryl, and
R5 is hydrogen, sulfonic acid or a sulfonic acid derivative,
R6 is hydrogen, bromine, chlorine, hydroxy, alkoxy, aryloxy or NR29R30 wherein R29 and R30 are each independently of the other hydrogen, alkyl, aryl or a reactive group A bound via a linker L, wherein R29 and R30 are each independently of the other preferably selected from (i) C₁-C₄ alkyl, (ii) ω-sulfonic acid alkyl (—(CH₂)ₓ—SO₃⁻) wherein x is 1-5, (iii) ω-carboxylic acid alkyl (—(CH₂)_{y}—CO₂H) wherein y is 1-8, and (iv) ethyl esters of (iii),
R7 is hydrogen, bromine, chlorine, hydroxy, alkoxy, aryloxy, NR31R32, sulfonic acid or a sulfonic acid derivative, wherein R31 and R32 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, (vii) ω-sulfonic acid alkyl (—(CH₂)ₓ—SO₃⁻) where x is 1-5, (viii) ω-carboxylic acid alkyl (—(CH₂)_{y}—CO₂H) where y is 1-8, and (ix) ethyl esters of (viii),
R8 is hydrogen, methyl or ethyl,
R9 is hydrogen, alkyl or 2-carboxyphenyl, and
L is a linker selected from —(CH₂)ₛ— and —[(CH₂)ₘ—O]ₚ—(CH₂)ₘ—, where m is an integer from 2-5 and p and s are each independently of the other an integer from 1-10, wherein
each compound contains no or one linker L having a reactive group A bound to L for covalently bonding to a molecule K to be labelled, wherein
A is an amine (—NH₂), hydroxy (—OH) or phosphoramidite (—O—P—[O—CH₂—CH₂—CN]—N[(CH(CH₃)₂]₂) function, a carboxylic acid (—COOH), an alkyl ester or active ester derived therefrom (NHS ester, sulfo-NHS ester, tetrafluoro-phenyl ester, p-sulfo-tetrafluoro-phenyl ester), a carboxylic acid hydrazide (—CONHNH₂) or a carboxylic acid amide (—CONHR28) with R28 equal to —(CH₂)ₜ—Y, where
Y is —OH, —NH₂, —NH₃⁺, maleimide (—N[CO—CH]₂), —NCS, —NCO, —NH—CO—CH₂—I, —NH—CO—CH₂—Br, -azide (—N₃), -alkyne (—CCH) or -phosphoramidite (—O—P—[O—CH₂—CH₂—CN]—N—[CH—(CH₃)₂]₂) and t is an integer from 1-10,
K is a component selected from the group haptens (molecules which represent an incomplete antigen and exhibit the effect of an antigen only when bound to proteins or cell structures), proteins, antibodies (proteins which are formed in response to antigens), low-molecular-weight drugs (active constituents in drugs which, because of their relatively low molar mass of up to about 800 g/mol, in contrast to, for example, proteins as very large molecules, are able to penetrate into cells), peptides (small or short-chain proteins up to about 100 linked amino acids), nucleotides (basic building blocks of nucleic acids such as DNA or RNA, which consist of a phosphate part, a monosaccharide part and a nucleobase part such as adenine, guanine, cytosine, thymine or uracil), nucleosides (basic building blocks of nucleic acids such as DNA or RNA, which do not have a phosphate part, but consist only of a monosaccharide part and a nucleobase part), DNA oligomers (in contrast to DNA as a macromolecule, molecules of deoxyribonucleic acid with a relatively small, not exactly defined number of nucleotides), polymers (synthetic or natural, chain-like or branched chemical compound consisting of repeating units, the monomers; polymers may also as copolymers consist of at least two different monomers in different proportions and arrangements).

### 2. Aspect

Compound according to aspect 1, characterized by

R3=hydroxy, wherein the compound, depending on the pH, is present as the neutral base structure 3-oxo-2H-xanthene 4

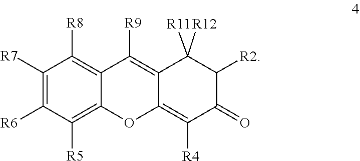

### 3. Aspect

Compound according to aspect 1 or 2, characterized in that

at least one selected from R2-R3, R3-R4, R5-R6, R6-R7 and R7-R8 is bridged by forming saturated rings, partially unsaturated rings, aromatic rings or heteroaromatic rings which independently of the other contain further substituents, in particular sulfonic acids or sulfonic acid derivatives.

### 4. Aspect

Compound according to any one of the preceding aspects, characterised by a structure of formula 5,

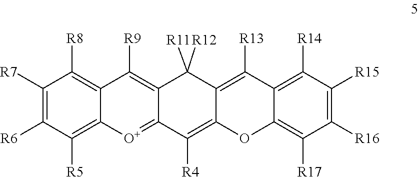

wherein
R13 is hydrogen, alkyl or 2-carboxyphenyl,
R14 is hydrogen, alkyl or 2-carboxyphenyl,
R15 is hydrogen, bromine, chlorine, hydroxy, alkoxy, aryloxy, NR33R34, sulfonic acid or a sulfonic acid derivative or bridged to R16, wherein R33 and R34 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, (vii) ω-sulfonic acid alkyl (—(CH₂)ₓ—SO₃⁻) where x is 1-5, (viii) ω-carboxylic acid alkyl (—(CH₂)_{y}—CO₂H) where y is 1-8, and (ix) ethyl esters of (viii),
R16 is hydrogen, bromine, chlorine, hydroxy, alkoxy, aryloxy or NR35R36, wherein R35 and R36 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, (vii) ω-sulfonic acid alkyl (—(CH₂)ₓ—SO₃⁻) where x is 1-5, (viii) ω-carboxylic acid alkyl (—(CH₂)_{y}—CO₂H) where y is 1-8, and (ix) ethyl esters of (viii),
R17 is hydrogen, sulfonic acid or a sulfonic acid derivative, and
one or more selected from R5-R6, R6-R7, R7-R8, R14-R15, R15-R16 and R16-R17 may be bridged by forming saturated rings, partially unsaturated rings, aromatic rings or heteroaromatic rings which may independently of the other contain further substituents, in particular sulfonic acids or sulfonic acid derivatives, and
preferably aryl substituents and/or heteroaryl substituents (as in R4, R6, R7, R15, R16, R18, R19, R33, R34, R35, R36) contain further substituents such as sulfonic acids or sulfonic acid derivatives and/or alkoxy groups and/or substituted amino groups.

### 5. Aspect

Compound according to any one of the preceding aspects, characterized in that the compound contains at least one sulfonic acid group.

### 6. Aspect

Compound according to any one of the preceding aspects, characterized in that R3 is NR18R19, wherein R18 and R19 are each independently of the other hydrogen, alkyl, benzyl, aryl, heteroaryl or a reactive group A bound via a linker L, wherein NR18R19 is preferably selected from 3-aminopropane sulfonate, N-methylaniline, 4-(methylamino)benzenesulfonate, aniline, 5-carboxypentylamine and 3-carboxypropyl(methyl)amine.

### 7. Aspect

Compound according to any one of the preceding aspects, characterised in that at least 1 mg of the compound is soluble in 1,000 g of water at 25° C.

### 8. Aspect

Compound according to any one of the preceding aspects, characterized in that the compound is a fluorescent compound having an absorption maximum in the wavelength range from 400 nm to 650 nm, preferably from 500 nm to 550 nm.

### 9. Aspect

Compound according to aspect 8, characterized in that the Stokes shift is at least 40 nm, preferably from 50 nm to 120 nm, more preferably from 70 nm to 90 nm.

### 10. Aspect

Compound according to any one of the preceding aspects, characterized in that the compound is selected from
6-ethoxy-N,N-diethyl-8,8-dimethyl-7H-xanthene-10-ium-3-amin,
6-[[6-(diethylamino)-1,1-dimethyl-2H-xanthene-10-ium-3-yl]amino]hexanoic acid,
3-[(6-ethoxy-8,8-dimethyl-7H-xanthene-10-ium-3-yl)-(6-ethoxy-6-oxo-hexyl)amino]propane-1-sulfonate,
3-[[6-[5-carboxypentyl(3-sulfonatopropyl)amino]-1,1-dimethyl-2H-xanthene-10-ium-3-yl]amino]propane-1-sulfonate,
3-[[6-[[6-(2,5-dioxopyrrolidine-1-yl)oxy-6-oxo-hexyl]-(3-sulfonatopropyl)amino]-1,1-dimethyl-2H-xanthene-10-ium-3-yl]amino]propane-1-sulfonate,
6-[(6-ethoxy-8,8-dimethyl-7H-xanthene-10-ium-3-yl)-ethyl-amino]hexanoic acid ethyl ester,
3-[[6-[5-carboxypentyl(ethyl)amino]-1,1-dimethyl-2H-xanthene-10-ium-3-yl]amino]propane-1-sulfonate,
3-[5-carboxypentyl-[8,8-dimethyl-6-(N-methylanilino)-7H-xanthene-10-ium-3-yl]amino]propane-1-sulfonate,
4-[[6-[5-carboxypentyl(3-sulfonatopropyl)amino]-1,1-dimethyl-2H-xanthene-10-ium-3-yl]-methyl-amino]benzenesulfonate,
6-[[8,8-dimethyl-6-(N-methylanilino)-7H-xanthene-10-ium-3-yl]-ethyl-amino]hexanoic acid,
4-[[6-[5-carboxypentyl(ethyl)amino]-1,1-dimethyl-2H-xanthene-10-ium-3-yl]-methyl-amino]benzenesulfonate,
6-[(6-anilino-8,8-dimethyl-7H-xanthene-10-ium-3-yl)-ethyl-amino]hexanoic acid,
6-[5-carboxypentyl(ethyl)amino]-1,1-dimethyl-3-(4-sulfonatoanilino)-2H-xanthene-10-ium-4-sulfonate,
6-[[6-[5-carboxypentyl(ethyl)amino]-1,1-dimethyl-2H-xanthene-10-ium-3-yl]amino]naphthalene-2-sulfonate,
6-ethoxy-8,8-dimethyl-7H-xanthene-10-ium-3-ol,
8,8-dimethyl-6-(N-methylanilino)-7H-xanthene-10-ium-3-ol,
6-[[8,8-dimethyl-6-(N-methylanilino)-7H-xanthene-10-ium-3-yl]oxy]hexanoic acid,
3-(5-carboxypentoxy)-8,8-dimethyl-6-(N-methyl-4-sulfonato-anilino)-7H-xanthene-10-ium-2-sulfonate,
3-(9-ethoxy-2,2,4,7,7-pentamethyl-8H-chromeno[3,2-g]quinolin-11-ium yl)propane-1-sulfonate,
6-(9-ethoxy-2,2,4,7,7-pentamethyl-8H-chromeno[3,2-g]quinolin-11-ium-1-yl)hexanoic acid ethyl ester,
3-[9-(5-carboxypentylamino)-2,2,4,7,7-pentamethyl-8H-chromeno[3,2-g]quinolin-11-ium-1-yl]propane-1-sulfonate,
3-[9-[3-carboxypropyl(methyl)amino]-2,2,4,7,7-pentamethyl-8H-chromeno[3,2-g]quinolin-11-ium-1-yl]propane-1-sulfonate,
6-[2,2,4,7,7-pentamethyl-9-(N-methylanilino)-8H-chromeno[3,2-g]quinolin-11-ium-1-yl]hexanoic acid,
4-[[1-(5-carboxypentyl)-2,2,7,7-tetramethyl-4-(sulfonatomethyl)-8H-chromeno[3,2-g]quinolin-11-ium-9-yl]-methyl-amino]benzenesulfonate,
6-[[6-[3-(dimethylamino)anilino]-8,8-dimethyl-7H-xanthene-10-ium-3-yl]-ethyl-amino]hexanoic acid,
6-[[6-(4-aminoanilino)-8,8-dimethyl-7H-xanthene-10-ium-3-yl]-ethyl-amino]hexanoic acid,
6-[[6-[bis(2-pyridylmethyl)amino]-8,8-dimethyl-7H-xanthene-10-ium-3-yl]-ethyl-amino]hexanoic acid,
6-[[8,8-dimethyl-6-[(E)-1H-pyridine-2-ylidenemethyl]-7H-xanthene-10-ium-3-yl]-ethyl-amino]hexanoic acid,
6-[[6-[4-(dimethylamino)phenyl]-8,8-dimethyl-7H-xanthene-10-ium-3-yl]-ethyl-amino]hexanoic acid,
6-[ethyl-(6,8,8-trimethyl-7H-xanthene-10-ium-3-yl)amino]hexanoic acid,
(2E)-1-(5-carboxypentyl)-2-[(E)-3-[6-(diethylamino)-1,1-dimethyl-2H-xanthene-10-ium-3-yl]prop-2-enylidene]-3,3-dimethyl-indoline-5-sulfonate,
3-[(5Z)-3-(5-carboxypentyl)-5-[(2E)-2-[6-(diethylamino)-1,1-dimethyl-2H-xanthene-3-ylidene]ethylidene]-2,4,6-trioxo-hexahydropyrimidine-1-yl]propane-1-sulfonate,
6-(diethylamino)-1,1-dimethyl-2H-xanthene-3-one,
6-[(8,8-dimethyl-6-oxo-7H-xanthene-3-yl)-ethyl-amino]hexanoic acid,
6-[5-carboxypentyl(ethyl)amino]-1,1-dimethyl-3-oxo-2H-xanthene-4-sulfonate,
3-[5-carboxypentyl(8,8-dimethyl-6-oxo-7H-xanthene-3-yl)amino]propane-1-sulfonate,
6-[5-carboxypentyl(3-sulfonatopropyl)amino]-1,1-dimethyl-3-oxo-2H-xanthene-4-sulfonate,
6-hydroxy-1,1-dimethyl-3-oxo-2H-xanthene-4-sulfonate,
6-(5-carboxypentoxy)-1,1-dimethyl-3-oxo-2H-xanthene-4-sulfonate,
6-(2,2,4,7,7-pentamethyl-9-oxo-8H-chromeno[3,2-g]quinolin-1-yl)hexanoic acid,
1-(5-carboxypentyl)-2,2,4,7,7-pentamethyl-9-oxo-8H-chromeno[3,2-g]quinoline sulfonate,
6-[[8,8-dimethyl-6-oxo-5-(4-pyridyl)-7H-xanthene-3-yl]-ethyl-amino]hexanoic acid,
3-[5-carboxypentyl-[8,8-dimethyl-6-oxo-5-(4-pyridyl)-7H-xanthene-3-yl]amino]propane-1-sulfonate,
2-[3,9-bis(diethylamino)-13,13-dimethyl-chromeno[3,2-b]xanthene-5-ium-14-yl]benzoic acid,
6-[[9-(diethylamino)-13,13-dimethyl-chromeno[3,2-b]xanthene-7-ium-3-yl]-ethyl-amino]hexanoic acid,
   und
6-[ethyl-(3-methoxy-13,13-dimethyl-chromeno[3,2-b]xanthene-5-ium-9-yl)amino]hexanoic acid.

### 11. Aspect

Method for preparing a compound of Formula 1, wherein the method comprises the reaction of (E)-(3-ethoxy-5,5-dimethyl-cyclohex-2-en-1-ylidene)ethyl-oxonium or (E)-[3-ethoxy-5,5-dimethyl-2-(4-pyridyl)cyclohex-2-en-1-ylidene]-ethyl-oxonium with a benzaldehyde compound, wherein the benzaldehyde compound has a benzaldehyde group and the benzaldehyde compound has a hydroxy group in ortho-position to the benzaldehyde group.

### 12. Aspect

Method for preparing a compound of Formula 5, wherein the method comprises the reaction of a compound of Formula 1, preferably 6-ethoxy-N,N-diethyl-8,8-dimethyl-7H-xanthene-10-ium-3-amine or 6-[(6-ethoxy-8,8-dimethyl-7H-xanthene-10-ium-3-yl)-ethyl-amino]hexanoic acid ethyl ester, with a benzaldehyde compound, wherein the benzaldehyde compound has a benzaldehyde group and the benzaldehyde compound has a hydroxy group in ortho-position to the benzaldehyde group.

### 13. Aspect

Use of a compound according to any one of aspects 1 to 10 as a fluorescent dye and/or in a fluorescent probe or as a fluorescent probe.

### 14. Aspect

Use according to aspect 13 for labelling one or more compounds selected from amino acids, peptides, proteins, antibodies, antigens, haptens, enzyme substrates, enzyme cofactors, biotin, carotenoids, hormones, neurohormones, neurotransmitters, growth factors, lectins, toxins, carbohydrates, oligosaccharides, polysaccharides, dextrans, nucleic acids, oligonucleotides, DNA, RNA, cells, lipids, receptor-binding pharmaceuticals.

### 15. Aspect

Use according to aspect 13 or 14 in optical, in particular fluorescence-optical, qualitative and/or quantitative determination methods for the diagnosis of cell properties, in biosensors (point of care measurements), for researching a genome (DNA sequencing), in cytometry and cell sorting, fluorescence correlation spectroscopy (FCS), ultra-high throughput screening (UHTS), multicolour fluorescence in situ hybridisation (FISH) and in microarrays (DNA and protein chips).

1. A compound of the general formula 1 or 4 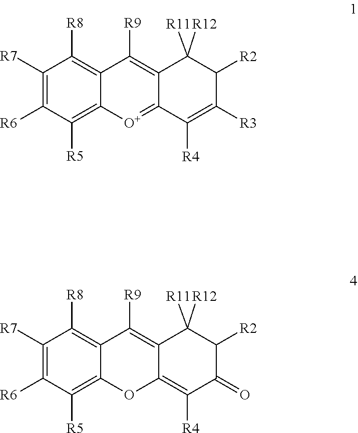
and salts as well as solvates thereof, wherein
R11 and R12 are each independently of the other hydrogen or alkyl,
R2 is hydrogen, alkyl or alkenyl,
R3 is hydrogen, alkyl, aryl, hydroxy, alkoxy, aryloxy, NR18R19 or a group Q,
wherein R18 and R19 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, more preferably ethyl, (vii) ω-sulfonic acid alkyl (—(CH₂)ₓ—SO₃⁻) where x is 1-5, (viii) ω-carboxylic acid alkyl (—(CH₂)_{y}—CO₂H) where y is 1-8, and (ix) ethyl esters of (viii),
and Q is a heterocyclic structure selected from a structure of formula 2 or 3 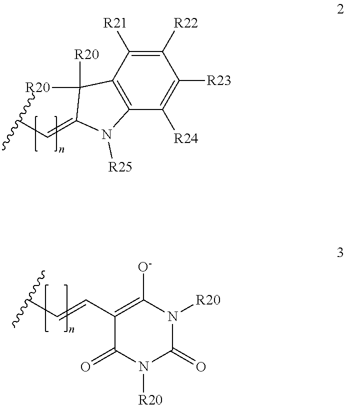
where n=1, 2 or 3; wherein each R20 is independently of the other alkyl, ω-sulfonic acid alkyl (—(CH₂)ₓ—SO₃⁻) or a reactive group A bound through a linker L; R21, R22, R23, R24 are independently of the other each hydrogen, a sulfonic acid or a sulfonic acid derivative; R25 is hydrogen, alkyl, ω-sulfonic acid alkyl (—(CH₂)ₓ—SO₃⁻) or a reactive group A bound through a linker L, wherein each x is an integer from 1-5,
or R2 and R3 are bridged to form a saturated ring, partially unsaturated ring, aromatic ring or heteroaromatic ring together with the carbon atoms to which they are bound, which ring may contain further substituents,
R4 is hydrogen, bromine, chlorine, sulfonic acid or a sulfonic acid derivative, alkyl, aryl or heteroaryl
or R3 and R4 are bridged to form a saturated ring, partially unsaturated ring, aromatic ring or heteroaromatic ring together with the carbon atoms to which they are bound, which ring may contain further substituents, in particular a sulfonic acid or a sulfonic acid derivative,
R5 is hydrogen, sulfonic acid or a sulfonic acid derivative,
R6 is hydrogen, bromine, chlorine, hydroxy, alkoxy, aryloxy or NR29R30, wherein R29 and R30 are each independently of the other hydrogen, alkyl, aryl or a reactive group A bound via a linker L wherein R29 and R30 are each independently of the other preferably selected from (i) C₁-C₄ alkyl, (ii) ω-sulfonic acid alkyl (—(CH₂)ₓ—SO₃⁻) wherein x is 1-5, (iii) ω-carboxylic acid alkyl (—(CH₂)_{y}—CO₂H) wherein y is 1-8, and (iv) ethyl esters of (iii),
or R5 and R6 are bridged to form a saturated ring, partially unsaturated ring, aromatic ring or heteroaromatic ring together with the carbon atoms to which they are bound, which ring may contain further substituents, in particular a sulfonic acid or sulfonic acid derivative,
R7 is hydrogen, bromine, chlorine, hydroxy, alkoxy, aryloxy, NR31R32, sulfonic acid or a sulfonic acid derivative, wherein R31 and R32 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, (vii) ω-sulfonic acid alkyl (—(CH₂)ₓ—SO₃⁻) where x is 1-5, (viii) ω-carboxylic acid alkyl (—(CH₂)_{y}—CO₂H) where y is 1-8, and (ix) ethyl esters of (viii),
or R6 and R7 are bridged to form a saturated ring, partially unsaturated ring, aromatic ring or heteroaromatic ring together with the carbon atoms to which they are bound, which ring may contain further substituents, in particular a sulfonic acid or sulfonic acid derivative,
R8 is hydrogen, methyl or ethyl,
or R7 and R8 are bridged to form a saturated ring, partially unsaturated ring, aromatic ring or heteroaromatic ring together with the carbon atoms to which they are bound, which ring may contain further substituents, in particular a sulfonic acid or a sulfonic acid derivative,
R9 is hydrogen, alkyl or 2-carboxyphenyl,
L is a linker selected from —(CH₂)ₛ— and —[(CH₂)ₘ—O]ₚ—(CH₂)ₘ—, where m is an integer from 2-5 and p and s are each independently of the other an integer from 1-10, wherein
the compound contains a linker L having a reactive group A bonded to L for covalently bonding to a molecule K to be labelled, wherein
A is an amine (—NH₂), hydroxy (—OH) or phosphoramidite (—O—P—[O—CH₂—CH₂—CN]—N[(CH(CH₃)₂]₂) function, a carboxylic acid (—COOH), an alkyl ester or active ester derived therefrom (NHS ester, sulfo-NHS ester, tetrafluoro-phenyl ester, p-sulfo-tetrafluoro-phenyl ester), a carboxylic acid hydrazide (—CONHNH₂) or a carboxylic acid amide (—CONHR28) with R28 equal to —(CH₂)ₜ—Y, where
Y is —OH, —NH₂, —NH₃⁺, maleimide (—N[CO—CH]₂), —NCS, —NCO, —NH—CO—CH₂—I, —NH—CO—CH₂—Br, -azide (—N₃), -alkyne (—CCH) or -phosphoramidite (—O—P—[O—CH₂—CH₂—CN]—N—[CH—(CH₃)₂]₂) and t is an integer from 1-10,
K is a component selected from the group consisting of haptens, proteins, antibodies, low-molecular weight drugs, peptides, nucleotides, nucleosides, DNA oligomers, polymers.

2. A compound according to claim 1, wherein R3=hydroxy.

3. A compound according to claim 1, wherein
R3 and R4 are not bridged together in such a way that they form an aromatic ring together with the carbon atoms to which they are bound.

4. A compound according to claim 1, comprising:
a structure of formula 5, 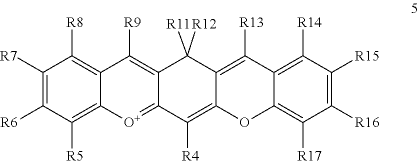
wherein
R13 is hydrogen, alkyl or 2-carboxyphenyl,
R14 is hydrogen, alkyl or 2-carboxyphenyl,
R15 is hydrogen, bromine chlorine, hydroxy, alkoxy, aryloxy, NR33R34, sulfonic acid or a sulfonic acid derivative or bridged to R16, wherein R33 and R34 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, (vii) ω-sulfonic acid alkyl (—(CH₂)ₓ—SO₃⁻) where x is 1-5, (viii) ω-carboxylic acid alkyl (—(CH₂)_{y}—CO₂H) where y is 1-8, and (ix) ethyl esters of (viii),
or R14 and R15 are bridged to form a saturated ring, partially unsaturated ring, aromatic ring or heteroaromatic ring together with the carbon atoms to which they are bound, which ring may contain further substituents, in particular a sulfonic acid or sulfonic acid derivative,
R16 is hydrogen, bromine, chlorine, hydroxy, alkoxy, aryloxy or NR35R36, where R35 and R36 are each independently of the other selected from (i) hydrogen, (ii) benzyl, (iii) aryl, (iv) heteroaryl, (v) a reactive group A bound via a linker L, (vi) alkyl, preferably C₁-C₄ alkyl, (vii) ω-sulfonic acid alkyl (—(CH₂)ₓ—SO₃⁻), where x is 1-5, (viii) ω-carboxylic acid alkyl (—(CH₂)_{y}—CO₂H), where y is 1-8, and (ix) ethyl esters of (viii),
or R15 and R16 are bridged to form a saturated ring, partially unsaturated ring, aromatic ring or heteroaromatic ring together with the carbon atoms to which they are bound, which ring may contain further substituents, in particular a sulfonic acid or sulfonic acid derivative,
R17 is hydrogen, sulfonic acid or a sulfonic acid derivative,
or R16 and R17 are bridged to form a saturated ring, partially unsaturated ring, aromatic ring or heteroaromatic ring together with the carbon atoms to which they are bound, which ring may contain further substituents, in particular a sulfonic acid or a sulfonic acid derivative.

5. A compound according to claim 1, wherein
the compound contains at least one group selected from a sulfonic acid group, a sulfonic acid derivative, a hydroxy group, an amino group, a carboxylic acid and a carboxylic acid derivative, wherein preferably compounds are excluded in which
a) R7 is methoxy and R3 is an amino group, in particular NR18R19, in particular NH(4-HOOCC₆H₄), NH(4-C₂H₅COOC₆H₄), NH(2-C₂H₅COOC₆H₄), NH(C₆H₅), N(C₂H₅)₂ or N(CH₂)₂(CH₂)₂O;
b) R4 is bromine and R6 and R16 are N(CH₃)₂;
c) R6 and R16 are present and one of R6 and R16 is a hydroxy group.

6. A compound according to claim 1, wherein the compound contains at least one sulfonic acid group.

7. A compound according to claim 1, wherein R3 is NR18R19, wherein R18 and R19 are each independently of the other hydrogen, alkyl, benzyl, aryl, heteroaryl or a reactive group A bound via a linker L, wherein NR18R19 is preferably selected from 3-aminopropanesulfonate, N-methylaniline, 4-(methylamino)benzenesulfonate, aniline, 5-carboxypentylamine and 3-carboxypropyl(methyl)amine.

8. A compound according to claim 1, wherein the compound is water soluble, preferably that at least 1 mg of the compound is soluble in 1000 mg of water at 25° C.

9. A compound according to claim 1, wherein the compound is a fluorescent compound having an absorption maximum in the wavelength range from 400 nm to 650 nm.

10. A compound according to claim 8, wherein the Stokes shift is at least 40 nm.

11. A compound according to claim 1, wherein the compound is selected from
6-ethoxy-N,N-diethyl-8,8-dimethyl-7H-xanthene-10-ium-3-amine,
6-[[6-(diethylamino)-1,1-dimethyl-2H-xanthene-10-ium-3-yl]amino]hexanoic acid,
3-[(6-ethoxy-8,8-dimethyl-7H-xanthene-10-ium-3-yl)-(6-ethoxy-6-oxo-hexyl)amino]propane-1-sulfonate,
3-[[6-[5-carboxypentyl(3-sulfonatopropyl)amino]-1,1-dimethyl-2H-xanthene ium-3-yl]amino]propane-1-sulfonate,
3-[[6-[[6-(2,5-dioxopyrrolidine-1-yl)oxy-6-oxo-hexyl]-(3-sulfonatopropyl)amino]-1,1-dimethyl-2H-xanthene-10-ium-3-yl]amino]propane-1-sulfonate,
6-[(6-ethoxy-8,8-dimethyl-7H-xanthene-10-ium-3-yl)-ethyl-amino]hexanoic acid ethyl ester,
3-[[6-[5-carboxypentyl(ethyl)amino]-1,1-dimethyl-2H-xanthene-10-ium-3-yl]amino]propane-1-sulfonate,
3-[5-carboxypentyl-[8,8-dimethyl-6-(N-methylanilino)-7H-xanthene-10-ium-3-yl]amino]propane-1-sulfonate,
4-[[6-[5-carboxypentyl(3-sulfonatopropyl)amino]-1,1-dimethyl-2H-xanthene-10-ium-3-yl]-methyl-amino]benzenesulfonate,
6-[[8,8-dimethyl-6-(N-methylanilino)-7H-xanthene-10-ium-3-yl]-ethyl-amino]hexanoic acid,
4-[[6-[5-carboxypentyl(ethyl)amino]-1,1-dimethyl-2H-xanthene-10-ium-3-yl]-methyl-amino]benzenesulfonate,
6-[(6-anilino-8,8-dimethyl-7H-xanthene-10-ium-3-yl)-ethyl-amino]hexanoic acid,
6-[5-carboxypentyl(ethyl)amino]-1,1-dimethyl-3-(4-sulfonatoanilino)-2H-xanthene-10-ium-4-sulfonate,
6-[[6-[5-carboxypentyl(ethyl)amino]-1,1-dimethyl-2H-xanthene-10-ium-3-yl]amino]naphthalene-2-sulfonate,
6-ethoxy-8,8-dimethyl-7H-xanthene-10-ium-3-ol,
8,8-dimethyl-6-(N-methylanilino)-7H-xanthene-10-ium-3-ol,
6-[[8,8-dimethyl-6-(N-methylanilino)-7H-xanthene-10-ium-3-yl]oxy]hexanoic acid,
3-(5-carboxypentoxy)-8,8-dimethyl-6-(N-methyl-4-sulfonato-anilino)-7H-xanthene-10-ium-2-sulfonate,
3-(9-ethoxy-2,2,4,7,7-pentamethyl-8H-chromeno[3,2-g]quinolin-11-ium-1-yl)propane-1-sulfonate,
6-(9-ethoxy-2,2,4,7,7-pentamethyl-8H-chromeno[3,2-g]quinolin-11-ium-1-yl)hexanoic acid ethyl ester,
3-[9-(5-carboxypentylamino)-2,2,4,7,7-pentamethyl-8H-chromeno[3,2-g]quinolin-11-ium-1-yl]propane-1-sulfonate,
3-[9-[3-carboxypropyl(methyl)amino]-2,2,4,7,7-pentamethyl-8H-chromeno[3,2-g]quinolin-11-ium-1-yl]propane-1-sulfonate,
6-[2,2,4,7,7-pentamethyl-9-(N-methylanilino)-8H-chromeno[3,2-g]quinolin-11-ium-1-yl]hexanoic acid,
4-[[1-(5-carboxypentyl)-2,2,7,7-tetramethyl-4-(sulfonatomethyl)-8H-chromeno[3,2-g]quinolin-11-ium-9-yl]-methyl-amino]benzenesulfonate,
6-[[6-[3-(dimethylamino)anilino]-8,8-dimethyl-7H-xanthene-10-ium-3-yl]-ethyl-amino]hexanoic acid,
6-[[6-(4-aminoanilino)-8,8-dimethyl-7H-xanthene-10-ium-3-yl]-ethyl-amino]hexanoic acid,
6-[[6-[bis(2-pyridylmethyl)amino]-8,8-dimethyl-7H-xanthene-10-ium-3-yl]-ethyl-amino]hexanoic acid,
6-[[8,8-dimethyl-6-[(E)-1H-pyridine-2-ylidenemethyl]-7H-xanthene-10-ium-3-yl]-ethyl-amino]hexanoic acid,
6-[[6-[4-(dimethylamino)phenyl]-8,8-dimethyl-7H-xanthene-10-ium-3-yl]-ethyl-amino]hexanoic acid,
6-[ethyl-(6,8,8-trimethyl-7H-xanthene-10-ium-3-yl)amino]hexanoic acid,
(2E)-1-(5-carboxypentyl)-2-[(E)-3-[6-(diethylamino)-1,1-dimethyl-2H-xanthene-10-ium-3-yl]prop-2-enylidene]-3,3-dimethyl-indoline-5-sulfonate,
3-[(5Z)-3-(5-carboxypentyl)-5-[(2E)-2-[6-(diethylamino)-1,1-dimethyl-2H-xanthene-3-ylidene]ethylidene]-2,4,6-trioxo-hexahydropyrimidine-1-yl]propane-1-sulfonate,
6-(diethylamino)-1,1-dimethyl-2H-xanthene-3-one,
6-[(8,8-dimethyl-6-oxo-7H-xanthene-3-yl)-ethyl-amino]hexanoic acid,
6-[5-carboxypentyl(ethyl)amino]-1,1-dimethyl-3-oxo-2H-xanthene-4-sulfonate,
3-[5-carboxypentyl(8,8-dimethyl-6-oxo-7H-xanthene-3-yl)amino]propane sulfonate,
6-[5-carboxypentyl(3-sulfonatopropyl)amino]-1,1-dimethyl-3-oxo-2H-xanthene sulfonate,
6-hydroxy-1,1-dimethyl-3-oxo-2H-xanthene-4-sulfonate,
6-(5-carboxypentoxy)-1,1-dimethyl-3-oxo-2H-xanthene-4-sulfonate,
6-(2,2,4,7,7-pentamethyl-9-oxo-8H-chromeno[3,2-g]quinolin-1-yl)hexanoic acid,
1-(5-carboxypentyl)-2,2,4,7,7-pentamethyl-9-oxo-8H-chromeno[3,2-g]quinolin-10-sulfonate,
6-[[8,8-dimethyl-6-oxo-5-(4-pyridyl)-7H-xanthene-3-yl]-ethyl-amino]hexanoic acid,
3-[5-carboxypentyl-[8,8-dimethyl-6-oxo-5-(4-pyridyl)-7H-xanthene-3-yl]amino]propane-1-sulfonate,
2-[3,9-bis(diethylamino)-13,13-dimethyl-chromeno[3,2-b]xanthene-5-ium-14-yl]benzoic acid,
6-[[9-(diethylamino)-13,13-dimethyl-chromeno[3,2-b]xanthene-7-ium-3-yl]-ethyl-amino]hexanoic acid,
and
6-[ethyl-(3-methoxy-13,13-dimethyl-chromeno[3,2-b]xanthene-5-ium-9-yl)amino]hexanoic acid.

12. A compound according to claim 1, wherein the compound is selected from
3-[[6-[5-carboxypentyl(3-sulfonatopropyl)amino]-1,1-dimethyl-2H-xanthene-10-ium-3-yl]amino]propane-1-sulfonate,
4-[[6-[5-carboxypentyl(3-sulfonatopropyl)amino]-1,1-dimethyl-2H-xanthene-10-ium-3-yl]-methyl-amino]benzenesulfonate,
6-[5-carboxypentyl(ethyl)amino]-1,1-dimethyl-3-oxo-2H-xanthene-4-sulfonate,
6-[5-carboxypentyl(3-sulfonatopropyl)amino]-1,1-dimethyl-3-oxo-2H-xanthene sulfonate, and
6-(5-carboxypentoxy)-1,1-dimethyl-3-oxo-2H-xanthene-4-sulfonate.

13. A method for preparing a compound of formula 1 according to claim 1, which method comprises the reaction of (E)-(3-ethoxy-5,5-dimethyl-cyclohex-2-en-1-ylidene)-ethyl-oxonium or (E)-[3-ethoxy-5,5-dimethyl-2-(4-pyridyl)cyclohex-2-en-1-ylidene]-ethyl-oxonium with a benzaldehyde compound, wherein the benzaldehyde compound has a benzaldehyde group and the benzaldehyde compound has a hydroxy group in ortho-position to the benzaldehyde group.

14. A method for preparing a compound of formula 5 according to claim 1, which method comprises the reaction of a compound of formula 1, preferably of 6-ethoxy-N,N-diethyl-8,8-dimethyl-7H-xanthene-10-ium-3-amine or 6-[(6-ethoxy-8,8-dimethyl-7H-xanthene-10-ium-3-yl)-ethyl-amino]hexanoic acid ethyl ester, with a benzaldehyde compound, wherein the benzaldehyde compound has a benzaldehyde group and the benzaldehyde compound has a hydroxy group in ortho-position to the benzaldehyde group.

15. Use of a compound according to claim 1 as a fluorescent dye and/or in a fluorescent probe or as a fluorescent probe.
